SEQUENCE LISTING (33 sequences)

SEQ ID NO: 1 - protein, 332 aa, Homo sapiens
MPEGPLVRKFHHLVSPFVGQQVVKTGGSSKKLQPASLQSLWLQDTQVHGKKLFLRFDLDEEMGPPGSSPTPEPPQKEVQKEGAADPKQVGEPSGQKTLDGSSRSAELVPQGEDDSEYLERDAPAGDAGRWLRVSFGLFGSVWVNDFSRAKKANKRGDWRDPSPRLVLHFGGGGFLAFYNCQLSWSSSPVVTPTCDILSEKFHRGQALEALGQAQPVCYTLLDQRYFSGLGNIIKNEALYRAGIHPLSLGSVLSASRREVLVDHVVEFSTAWLQGKFQGRPQHTQVYQKEQCPAGHQVMKEAFGPEDGLQRLTWWCPQCQPQLSEEPEQCQFS

SEQ ID NO: 2 - protein, 49 aa, synthetic construct
  REGION 1..49: note = synthetic peptide
KKKKAAVALLPAVLLALLAPMSVLTPLLLRGLTGSARRLPVPRAKIHSL

SEQ ID NO: 3 - protein, 53 aa, synthetic construct
  REGION 1..53: note = synthetic peptide
KKKKKKKKAAVALLPAVLLALLAPMSVLTPLLLRGLTGSARRLPVPRAKIHSL

SEQ ID NO: 4 - protein, 57 aa, synthetic construct
  REGION 1..57: note = synthetic peptide
KKKKKKKKKKKKAAVALLPAVLLALLAPMSVLTPLLLRGLTGSARRLPVPRAKIHSL

SEQ ID NO: 5 - protein, 61 aa, synthetic construct
  REGION 1..61: note = synthetic peptide
KKKKKKKKKKKKKKKKAAVALLPAVLLALLAPMSVLTPLLLRGLTGSARRLPVPRAKIHSL

SEQ ID NO: 6 - protein, 20 aa, synthetic construct
  REGION 1..20: note = synthetic peptide
KKKKAAVALLPAVLLALLAP

SEQ ID NO: 7 - protein, 24 aa, synthetic construct
  REGION 1..24: note = synthetic peptide
KKKKKKKKAAVALLPAVLLALLAP

SEQ ID NO: 8 - protein, 28 aa, synthetic construct
  REGION 1..28: note = synthetic peptide
KKKKKKKKKKKKAAVALLPAVLLALLAP

SEQ ID NO: 9 - protein, 32 aa, synthetic construct
  REGION 1..32: note = synthetic peptide
KKKKKKKKKKKKKKKKAAVALLPAVLLALLAP

SEQ ID NO: 10 - protein, 33 aa, synthetic construct
  REGION 1..33: note = synthetic peptide
YKKKKKKKKKKKKKKKKAAVALLPAVLLALLAP

SEQ ID NO: 11 - protein, 48 aa, synthetic construct
  REGION 1..48: note = synthetic peptide
KKKKKKKKKKKKKKKKAAVALLPAVLLALLAPAAVALLPAVLLALLAP

SEQ ID NO: 12 - protein, 44 aa, synthetic construct
  REGION 1..44: note = synthetic peptide
AAVALLPAVLLALLAPKKKKKKKKKKKKAAVALLPAVLLALLAP

SEQ ID NO: 13 - protein, 32 aa, synthetic construct
  REGION 1..32: note = synthetic peptide
KKKKKKKKKKKKKKKKAAVWLLWYVLLFLLYL

SEQ ID NO: 14 - protein, 32 aa, synthetic construct
  REGION 1..32: note = synthetic peptide
KKKKKKKKKKKKKKKKFWVWLLWYVLLFLLYL

SEQ ID NO: 15 - protein, 4 aa, synthetic construct
  REGION 1..4: note = synthetic peptide
KKKK

SEQ ID NO: 16 - protein, 8 aa, synthetic construct
  REGION 1..8: note = synthetic peptide
KKKKKKKK

SEQ ID NO: 17 - protein, 12 aa, synthetic construct
  REGION 1..12: note = synthetic peptide
KKKKKKKKKKKK

SEQ ID NO: 18 - protein, 16 aa, synthetic construct
  REGION 1..16: note = synthetic peptide
KKKKKKKKKKKKKKKK

SEQ ID NO: 19 - protein, 4 aa, synthetic construct
  REGION 1..4: note = synthetic peptide
RRRR

SEQ ID NO: 20 - protein, 8 aa, synthetic construct
  REGION 1..8: note = synthetic peptide
RRRRRRRR

SEQ ID NO: 21 - protein, 12 aa, synthetic construct
  REGION 1..12: note = synthetic peptide
RRRRRRRRRRRR

SEQ ID NO: 22 - protein, 16 aa, synthetic construct
  REGION 1..16: note = synthetic peptide
RRRRRRRRRRRRRRRR

SEQ ID NO: 23 - protein, 4 aa, synthetic construct
  REGION 1..4: note = synthetic peptide
KRKR

SEQ ID NO: 24 - protein, 4 aa, synthetic construct
  REGION 1..4: note = synthetic peptide
KKKR

SEQ ID NO: 25 - protein, 12 aa, synthetic construct
  REGION 1..12: note = synthetic peptide
KKKRRRKKKRRR

SEQ ID NO: 26 - protein, 16 aa, synthetic construct
  REGION 1..16: note = synthetic peptide
KKKKRRRRKKKKRRRR

SEQ ID NO: 27 - protein, 7 aa, synthetic construct
  REGION 1..7: note = synthetic peptide
  PEPTIDE 1: note = X = V, I, L, M, F, W, C, A, Y, H, T, S, P, or G
  PEPTIDE 3: note = X = V, I, L, M, F, W, C, A, Y, H, T, S, P, or G
  PEPTIDE 7: note = X = V, I, L, M, F, W, C, A, Y, H, T, S, P, or G
XRXLRRX

SEQ ID NO: 28 - protein, 7 aa, synthetic construct
  REGION 1..7: note = synthetic peptide
PKKKRKV

SEQ ID NO: 29 - protein, 25 aa, synthetic construct
  REGION 1..25: note = synthetic peptide
LVRKKRKTEEESPLKDKDAKKSKQE

SEQ ID NO: 30 - protein, 12 aa, synthetic construct
  REGION 1..12: note = synthetic peptide
PEVKKKRKPEYP

SEQ ID NO: 31 - protein, 16 aa, synthetic construct
  REGION 1..16: note = synthetic peptide
  VARIANT 1: note = X = L, A, W, F, Y, or V
  VARIANT 2: note = X = L, A, W, F, Y, or V
  VARIANT 4: note = X = L, A, W, F, Y, or V
  VARIANT 7: note = X = L, A, W, F, Y, or V
  VARIANT 8: note = X = L, A, W, F, Y, or V
  VARIANT 12: note = X = L, A, W, F, Y, or V
  VARIANT 15: note = X = L, A, W, F, Y, or V
  VARIANT 16: note = X = L, A, W, F, Y, or V
XXVXLLXXVLLXLLXX

SEQ ID NO: 32 - protein, 16 aa, synthetic construct
  REGION 1..16: note = sytnhetic peptide
AAVALLPAVLLALLAP

SEQ ID NO: 33 - protein, 45 aa, synthetic construct
  REGION 1..45: note = synthetic peptide
AAVALLPAVLLALLAPMSVLTPLLLRGLTGSARRLPVPRAKIHSL

(19) United States
(12) Patent Application Publication
(10) Pub. No.: US 2023/0355725 A1
(43) Pub. Date: Nov. 9, 2023

(54) NEIL2 PROTEIN THERAPY FOR TREATMENT OF VIRAL INFECTION

(71) Applicant: Board of Regents, The University of Texas System, Austin, TX (US)

(72) Inventors: Tapas Hazra, Galveston, TX (US); Istvan Boldogh, Galveston, TX (US); Chien-Te K. Tseng, Galveston, TX (US)

(21) Appl. No.: 18/094,973

(22) Filed: Jan. 9, 2023

Related U.S. Application Data
(60) Provisional application No. 63/319,335, filed on Mar. 13, 2022.

(51) Int. Cl. A61K 38/51; A61K 9/00; A61K 47/50; A61P 31/14

(52) U.S. Cl. CPC A61K 38/51; C12Y 402/99018; A61K 9/0043; A61K 9/007; A61K 47/50; A61P 31/14; C07K 2319/33

(57) ABSTRACT

Certain embodiments are directed to methods for treating a virus induced dysregulated inflammatory response in a subject in need of such treatment. The methods can include the administration a NEIL2 peptide or a composition comprising the NEIL2 peptide to the subject.

### PRIORITY PARAGRAPH

This application claims priority to U.S. Provisional Patent Application Ser. No. 63/319,335 filed on Mar. 13, 2022 which is incorporated herein by reference in its entirety.

### STATEMENT REGARDING FEDERALLY FUNDED RESEARCH

This invention was made with government support under A1062885 awarded by the National Institutes of Health. The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

It is now widely accepted that inflammation underlies a wide variety of physiological and pathological processes, including disease caused by viral infections. Reactive oxygen species (ROS) generated due to dysregulated inflammatory and/or immune response are known to induce significant amount of DNA damage. Most of these DNA lesions are mutagenic and/or cytotoxic and have been implicated in a wide variety of pathophysiological states. Recent SARS-CoV-2 pandemic is posing a significant threat to both human health and global economy. No curative therapies for COVID-19 are available. Replicating SARS-CoV-2-induced dysregulated and often exacerbated inflammatory and/or immune responses also generate excessive amounts of reactive oxygen/nitrogen species (R/NS) that can cause damage to genomic DNA and activate DNA damage response pathway.

Currently, SARS-CoV-2 pandemic poses significant threat to both human health and global economy. No curative therapies for COVID-19 were available until recently (with FDA-approval of Pfizer's and others' anti-SARS-CoV-2 pills), but vaccines had been developed and widely used globally. However, emerging variants are still infecting fully vaccinated individuals causing illness, although with less severity. This is a vexing issue, particularly for RNA viruses containing single-strand genomes with high mutation rate. Thus, neutralizing monoclonal antibodies, an important biologic for treating viral infections may not be fully effective against mutated virus, as observed with the Omicron variant of SARS-CoV-2^{1,2}. Hence, development of new antiviral therapies beyond neutralizing antibodies is urgently needed to cope with the COVID-19 pandemic and future epidemic/pandemic threats as well.

The fact that SARS-CoV-2 induced dysregulated inflammatory and/or immune responses contribute to severe COVID-19 pathology make it an attractive target for therapeutic intervention and as proof of principle for other viruses with similar etiologies. The impact of elevated inflammatory responses on COVID-19 pathogenesis remains complex. In one instance disruption of IL6 (a major cytokine highly associated with severe COVID-19) signaling by administration of an antibody against IL-6R (tocilizumab) was reported to shorten the hospital stay of severe COVID-19 patients^{3,4}; unfortunately, however, a phase 3 trial did not validate the benefit of tocilizumab treatment against COVID-19^{5,6}. Additionally, several studies are examining the potential of anti-TNFα therapy as a treatment for COVID-19⁷⁻⁹. Anti-inflammatory drugs are commonly used for treating such diseases, but in many cases these treatments are associated with serious side effects^{7,10}, and thus cannot provide a long-term beneficial effect. Thus, there is a need for additional therapies for treating virus induced dysregulated inflammatory or immune response.

### SUMMARY

The inventors have developed solutions for the problems associated with the lack of therapeutic options for treating COVID-19 and other viral infections that produce or induce a dysregulated inflammatory or immune response. The solutions include methods and compositions for NEIL2-mediated amelioration of virus produced or virus induced dysregulated inflammatory or immune response, e.g., inhibiting viral replication and attenuation of elevated cytokine expression—providing additional strategies for treating virus produced or induced dysregulated inflammatory or immune response. Additionally, NEIL2 being a DNA repair protein has an advantage over other antiviral treatments as it can repair damage to the host genome caused by infectious agents and prevent further disease progression.

Certain aspects of embodiments described herein include but are not limited to (1) identifying at-risk human population groups that express lower levels of NEIL2 protein; (2) using a DNA repair protein in alleviating SARS-CoV-2 or other viral infection; (3) blocking exuberant immune-responses to diverse viruses and variants of concern providing compositions and methods for addressing future virus based threats; and (4) addressing the known issues associated with COVID-19 and other viral pandemics through development of technologies including non-invasive delivery of proteins as therapeutic agents to the lung. NEIL2 can ameliorate viral pathogenesis by decreasing host inflammatory response, inhibiting virus replication, and repairing host genome damage, thereby blocking or ameliorating disease severity. NEIL2 or compositions containing NEIL2 can be administered to a subject infected with a virus or a subject suspected of having a viral infection (e.g., elevated temperature etc.) or a subject at risk of being infected (e.g., subject is in a hot spot relative to viral infections (e.g., resides or is traveling into a location experiencing an outbreak, a seasonal outbreak, epidemic or pandemic). The traditional approaches for treating COVID-19 patients include prevention/inhibition of viral entry, prevention/inhibition of viral replication, or inhibition of resulting inflammation by specific cytokine inhibitors or nonspecific anti-inflammatory agents such as dexamethasone^{11,12}. NEIL2 possesses diverse functions in regulation of viral pathogenesis, both canonical (DNA repair) and non-canonical (anti-inflammatory and binding to 5′-UTR to block viral protein synthesis). These properties can be exploited in mitigating viral infection.

The lungs of a healthy adult subject negatively regulate the immune response. This negative regulation is reduced during a respiratory virus infection (Toapanta and Ross, 2009; Sharma and Goodwin, 2006) and can lead to a dysregulated inflammatory or immune response. Virus replication and delayed early innate immune response lead to dysregulation by over-activating immune cells to compensate for delayed or weak immune responses. Furthermore, release of Damage-Associated Molecular Patterns (DAMPs) by damaged cells leads to over-activation of cGAS-cGAMP-STING pathway and dysregulation of IFN-I production. This leads to an influx of pathogenic inflammatory monocyte-macrophages (IMMs) and neutrophils, which ultimately result in inflammation and severe damage to the lung and other tissues such as seen in COVID-19 (Channappanavar et al., 2016; Motwani et al., 2019; Liu et al., 2014). The dysregulation of the inflammatory or immune response leads to the release of large amounts of pro-inflammatory cytokines (IFN-α, IFN-γ, IL-1(3, IL-6, IL-12, IL-8, IL-18, IL-33, TNF-α, etc.) and chemokines (CCL2, CCL3, CCL5, CXCL8, CXCL9, CXCL10, etc.) and can lead to a “cytokine storm”. Inflammatory mediators produced by IMMs and neutrophils such as IL-6, IL-1β, and TNF-α, ROS and nitric oxide (NO) typically show more adverse effects than others. NO and ROS can enhance endothelial permeability and extravasation of immune cells into the lungs which lead to damage of alveolar epithelium, impair efficient gas exchange and finally respiratory distress (Short et al., 2014). Activated endothelium can secrete proinflammatory cytokines and chemokines such as CCL5/RANTES and IP-10/CXCL10 recruiting leukocytes that exacerbate inflammation and airway hyper-reactivity (Wang et al., 2013). This virus induced immune response leads to dysfunction in certain subjects.

Other aspects are directed to methods and protocols for protein delivery into the lung via noninvasive intranasal route for treating virus produced or induced dysregulated inflammatory or immune response, e.g., SARS-CoV-2 or RSV produced or induced dysregulated inflammatory or immune response. This multi-faceted therapeutic approach to combat dysregulated inflammatory responses, e.g., COVID-19, is an entirely new undertaking linking the axis of virus replication, inflammation, and DNA repair. The studies described herein are thus highly innovative.

Certain embodiments are directed to methods for treating a virus induced dysregulated inflammatory response in a subject in need of such treatment. The method comprising the step of administering to the subject a therapeutically effective amount of a NEIL2 peptide or a composition comprising the NEIL2 peptide, wherein the treatment inhibits and/or ameliorates at least one symptom of the virus induced dysregulated inflammatory response. In certain aspects the NEIL2 peptide or the composition comprising the NEIL2 peptide is administered by inhalation or instillation to the lungs. In a particular aspect the NEIL2 peptide or the composition comprising the NEIL2 peptide is administered intranasally. The NEIL2 peptide can have an amino acid sequence that is at least 80, 90, 95, to 100% identical to the amino acid sequence of SEQ ID NO:1. In certain aspects the NEIL2 peptide is a functional fragment of SEQ ID NO:1 including at least 70, 80, 90, 91, 92, 93, 94, 95, 96, 97, 98, or 99% of the amino acids of SEQ ID NO:1, preferably amino terminal or carboxy terminal fragment but can include a discontinuous fragment (i.e., internal deletions). The NEIL2 peptide can have the amino acid sequence of SEQ ID NO:1. In certain aspects the NEIL2 peptide is a fusion peptide or peptide complex comprising a targeting segment. In certain aspects the targeting segment is a lung cell targeting segment. The lung cell targeting segment can have an amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13. In certain aspects the lung cell targeting segment has an amino acid sequence of SEQ ID NO:9. The methods can further comprise a step of administering to the subject at least one additional antiviral therapeutic agent. In certain aspects the symptom is at least one of respiratory distress, organ dysfunction, systemic inflammatory response syndrome (SIRS), hypotension, tachycardia, dyspnea, ischemia, insufficient tissue perfusion, uncontrollable hemorrhage, multisystem organ failure or severe metabolism dysregulation. The virus can be from Coronaviridae, Pneumoviridae, Orthomyxoviridae, Filoviridae, Flaviviridae, or Poxviridae families. In certain aspects virus is a Coronavirus or a Respiratory syncytial virus (RSV). In certain aspects the Coronavirus is a SARS-CoV-2 virus. In certain aspects the subject is a human subject. The NEIL2 peptide can be administered to the subject at an amount from about 0.05 mg to about 0.5 mg peptide/kg body weight of said subject. The NEIL2 peptide and the additional antiviral therapeutic agent can be administered to the subject simultaneously, optionally co-formulated. In other aspects the NEIL2 peptide and the additional antiviral therapeutic agent are administered to the subject at different times.

Certain embodiments are directed to methods for treating a subject having a viral infection comprising the step of administering a therapeutically effective amount of a NEIL2 peptide or a composition comprising the NEIL2 peptide to the subject by inhalation or instillation. The method can further comprise identifying a subject at risk of a dysregulated immune response by measuring NEIL2 protein levels in a biological sample from the subject and identifying a subject to be treated when NEIL2 protein levels are below a reference level, normal, or threshold level for NEIL2 protein levels. In certain aspects the biological sample is peripheral blood mononuclear cells (PBMCs), bronhio-alovealar lavage fluid, or a lung biopsy. In certain aspects the virus is a Coronavirus or Pneumovirus. The Coronavirus can be a SARS-CoV-2 virus and the Pneumovirus can be a respiratory syncytial virus (RSV).

The “amount” or “level” of a protein or peptide is a detectable level in a biological sample. These can be measured by methods known to one skilled in the art and also disclosed herein. The expression level or amount of a protein or peptide assessed can be used to determine the response to the treatment or identify a subject in need of treatment.

The terms “level of expression” or “expression level” in general are used interchangeably and generally refer to the amount of a protein or peptide in a biological sample. “Expression” generally refers to the process by which information (e.g., gene-encoded and/or epigenetic) is converted into the structures present and operating in the cell. Therefore, as used herein, “expression” may refer to transcription into a polynucleotide and/or translation into a polypeptide. Fragments of the transcribed polynucleotide, the translated polypeptide, or polynucleotide shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a protein or peptide in an individual or sample from an individual relative to a control, such as an individual or individuals who are not suffering from the disease or disorder (e.g., viral infection) or an internal control (e.g., housekeeping gene/protein).

“Reduced expression,” “reduced expression levels,” or “reduced levels” refers to a decrease expression or decreased levels of a protein or peptide in an individual or sample from an individual relative to a control, such as an individual or individuals who are not suffering from the disease or disorder (e.g., viral infection) or an internal control (e.g., housekeeping gene/protein). In some embodiments, reduced expression is little or no expression.

The term “housekeeping gene or protein (gene/protein)” refers to a polynucleotide and/or polypeptide which are typically similarly present in all cell types. A “housekeeping gene” refers herein to a gene or group of genes which encode proteins whose activities are essential for the maintenance of cell function and which are typically similarly present in all cell types.

Other embodiments are directed to kits for the treatment of a viral pathogen infection and/or at least one symptom thereof in a human subject in need of such treatment, comprising: (a) at least one NEIL2 peptide and (b) a container containing at least on NEIL2 peptide. The kit can include instructions for use and/or a device (inhaler etc.) for administering the at least one NEIL2 peptide. The kit can also include reagents, e.g., NEIL2 antibody, for measuring the level of NEIL2 in a biological sample.

Other embodiments are directed to methods of treating a viral infection comprising: identifying a subject at risk for a virus induced dysregulation of inflammatory response by measuring NEIL2 protein levels in a biological sample from a subject suspected of such a risk, and administering a therapeutic composition comprising a NEIL2 peptide as described herein to the subject when the subject is identified as at risk for a virus induced dysregulation of inflammatory response when the NEIL2 protein levels are below a reference, normal, or threshold level for NEIL2 protein. In certain aspects the biological sample is a bronchio-alveolar fluid or peripheral blood mononuclear cells (PBMCs) or tissue sample (e.g., lung biopsy). The NEIL2 protein or mRNA levels can be at least or about 0.001, 0.01. 0.1, 1, 10, 20, 30, 40, or 50% of a reference or control (at lease 0.001×, 0.01×, 0.1×, 0.2×, 0.3×, 0.4×, or 0.5× of a control or reference level). In certain aspects a reference level can be established by averaging levels across non-diseased or unaffected subjects.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1C. (A) Heatmap showing changes in the expression among five oxidized base-specific DNA glycosylases in bronchoalveolar lavage fluid immune cells obtained from healthy controls (H) and patients with mild (M) and severe (S) COVID-19. (B) Expression level of NEIL2 transcript is significantly decreased in severe (S) COVID-19. (C) NEIL2 is downregulated in the lung after CoV-2 infection. Bar graphs display the levels of expression of NEIL2 (left) and OGG1 (right panel) in hamster lungs, as determined by qPCR.

FIG. 2A-2C. Immunohistochemical analysis of NEIL2 in the postmortem COVID-19 lung tissue. (A) Normal lung tissue obtained during surgical resection (top) or lung tissue obtained during autopsy studies on COVID-19 patients (bottom) were stained for NEIL2. Alv, alveoli. (B) Violin plots display the intensity of staining between normal lung and COVID-19 lung, as determined by IHC profiler. (C) Transcript level of NEIL2 in the PBMCs of healthy (n=4) vs moderate (n=3) and severe (n=8) COVID-19 patients hospitalized at UTMB.

FIG. 3A-3C. Low level of NEIL2 indicates a poor prognosis in COVID-19. Whisker plots display the expression levels of NEIL2 in a cohort of hospitalized patients, stratified based on their level of care (ICU vs. non-ICU) (A), and requirement of Mechanical Ventilator (MV vs. no-MV) (B) and diagnosis (COVID-19 vs. non-COVID-19). (C) Kaplan-Meier plots display the cumulative probability of discharge stratified based on high vs low levels of expression of NEIL2.

FIG. 4A-4B. NEIL2 protein transduction significantly inhibits viral replication of SARS-CoV-2 in A549-ACE2 cells. (A) Recombinant NEIL2, NEIL1 (1 μg per 10⁶ cells) transduced and mock transduced (control) A549-ACE2 cells were infected with SARS-CoV-2 (WA1-2020 at MOI 1) and supernatants were harvested at 24 h post-infection. Viral titer was measured using standard viral plaque assay with VeroE6 cells. NEIL2 transduced cells produced significantly less (* p<0.05) infectious virus compared to control and NEIL1 transduced cells. (B) NEIL2 mRNA expression in uninfected and CoV-2 infected mock, rNEIL1 and rNEIL2 transduced cells.

FIG. 5A-5B. (A) RNA ChIP-qPCR analysis in pcDNA3-CoV2-5′UTR-GFP transfected BEAS-2B cells. (B) RNA-EMSA showing binding of rNEIL2 to 45-mer RNA oligo containing ZnF sites (site-1 and -2) derived from 5′-UTR of CoV-2.

FIG. 6A-6B. NEIL2 overexpressing cells suppress GFP protein expression from a reporter plasmid under the control of 5′-UTR of Cov-2 (A). UTR less plasmid does not show such effect (B).

FIG. 7A-7B. (A) GFP reporter plasmid was constructed by inserting 300nt long 5′-UTR of CoV-2, and the internal ribosomal entry site (IRES) in the original plasmid was deleted as shown in the Figure. (B) Western analysis showing dose-dependent inhibition of GFP expression by rNEIL2 from a reporter plasmid under the control of 5′-UTR of CoV-2.

FIG. 8A-8B. Young-adult BALB/c mice were permissive to mouse-adapted (MA) SARS-CoV-2-MA10 infection. Five groups of 10 BALB/c mice (8-10 weeks old) were challenged (i.n.) with indicated amounts of MA10 virus or remained mock-infected and subjected to monitoring daily for the weight changes and accumulated mortality if any. A dose-dependent weight loss (A) and up to 30% of mortality (B) was observed.

FIG. 9A-9D. Neil2^{-/-} mice are highly susceptible to SARS-CoV-2 MA10 infection with increased mortality and lung viral load. Sixteen Neil2^{-/-} and 16 Neil2^{+/+} mice were challenged i.n. with 2×10⁵ TCID50 of CoV2 MA10. Ten mice were monitored for body weight change and mortality and 6 mice were euthanized at 2 dpi to assess the lung viral load by TCID50 assay. (A) Percent body weight change. (B) Percent survival plot showing the mortality. (C) Viral yield graph (TCID50/gm) of lung in Log10 scale. Limit of detection (LOD): limit of detection 1.45*103 TCID50/gm. (D) Western blot showing downregulation of NEIL2 in WT mice (n=4). ** p<0.005.

FIG. 10. Evaluation of DNA damage levels. LA-qPCR was performed in genomic DNA isolated from mice lung tissues 5 days post-infection. The bar diagram shows normalized relative PCR amplified band intensities of two representative genes in uninfected vs infected (MA10) WT mice lung tissues. n=3

FIG. 11A-11C. AC70 hACE2-Tg mice were permissive to wild-type SARS-CoV-2(US-WA1/2020) and all 4 major VOC strains, resulting in the onset of weight loss and mortality with different kinetics. Five groups of 8 AC70 hACE2-Tg mice were challenged (i.n.) with 5 TCID50 (˜1.7 LD50 of WA-1) of WA1/2020, α, β, γ, δ-variants, respectively. Five mice from each group were monitored for weight loss (A) and mortality (B). Three mice from each group were euthanized at 3 dpi and measured viral load in lungs and brain (C).

FIG. 12A-12C. BALB/c mice are permissive to β- and γ-variants of SARS-CoV-2 infection, resulting in weight loss, symptoms of illness and detected live virus in lungs. Five groups of 8 BALB/c mice were infected (i.n.) with 6×10⁵, 1.5×10⁴, 1.5×10⁵, 5.3×10⁴, and 3.5×10⁵ TCID50 of WA1/2020, α-, β-, γ-, and δ-variants of SARS-CoV-2, respectively. Five mice from each group were monitored for weight loss (A) and clinical score (B). Three mice from each group were euthanized at 2 dpi and measured viral load in lungs (C).

FIG. 13A-13B. Hamsters are permissive to SARS-CoV-2 with body weight loss and virus shedding in nasal swab. Hamsters (7-8-week-old) were challenged (i.n.) with 1×10⁶ TCID50 of SARS-CoV-2_WA1/2020 or mock-infected with PBS. Hamsters were monitored daily for changes in body weight. Average percent body weight change with SEM values (A). Dot plot showing the live virus yield in nasal swabs (B).

FIG. 14A-14F. (A) Increased mRNA levels of RSV NS1, N, G, F, and L in lungs of Neil2 KO mice compared to WT ones. (B) NEIL2 lowers level of viral genome. (C) NEIL2 is transiently down regulated in lungs of RSV-infected WT mice. (D) Percent body weight loss in WT vs Neil2 KO after RSV infection. (E, F) Prior administration of rNEIL2 (15 μg/mouse), via IN route, protects both Neil2-KO and WT from viral infection-induced weight loss.

FIG. 15A-15B. (A) Heatmap showing changes in expression of a subset of proinflammatory genes, associated with cytokine storm observed in COVID-19 patients, in Neil2^{-/-} vs. Neil2^{+/+} mice lung, post CoV2-MA10 infection. (B) Validation of PCR array data for expression of indicated genes by qPCR in CoV2-MA10 infected Neil2^{+/+} or Neil2^{-/-} mice lung relative to mock-treated Neil2^{+/+} mice lung. Results are normalized to 18S. Error bars represent ±SD of the mean. n=3 independent experiments from samples prepared by pooling lungs from n=3 mice per group.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

The expression of DNA repair genes in publicly available transcriptomic datasets were analyzed and found that the level of NEIL2, an oxidized base specific mammalian DNA glycosylase, is significantly low in patients who suffered from severe COVID-19 compared to the patients with milder disease. The correlation between NEIL2's level and other aspects of COVID-19 severity were then analyzed, such as requirements for ICU and mechanical ventilators (MV) among the cohorts of COVID-19 and non-COVID-19 patients. Unlike the levels of other DNA glycosylases, patients requiring ICU or MV had significantly lower levels of NEIL2 compared to non-ICU or non-MV patients. Moreover, a study involving 100 hospitalized COVID-19 patients showed a significant correlation between higher NEIL2 levels and shorter duration of hospitalization. Collectively these observations suggest a link between NEIL2 deficiency and COVID-19 severity. Of note, non-COVID patients with lower level of NEIL2 are also severely sick and they were admitted to ICU or needed mechanical ventilation, suggesting NEIL2's overall protective role.

The transcriptomic data also showed that NEIL2's level is significantly low in severely sick non-COVID patients suggesting other types of viral infection can also be severe in such patients. Respiratory pathogen RSV (BSL2 level RNA virus) can cause symptoms similar to SARS-CoV-2 in children, elderly and in specific risk groups of adults. Recent studies have indicated that 3-4% of COVID-19 patients also have co-infection with RSV. The inventors thus used it as a model and tested the effect of its infection on the DNA repair protein NEIL2. Neil2^{+/+} and Neil2^{-/-} mice were infected intranasally with 10⁶ PFU of purified RSV and lungs were excised at 24 h and 96 h after viral infection. Total RNAs were isolated, and viral mRNAs and genome levels were determined. It was found that the transcript levels of the viral genes (NS1, N, G, F and L) were increased by over 20-fold in both at 24 h and 96 h post RSV-challenge. Similar increase was also observed in the viral genomic levels in Neil2^{-/-} lungs. Moreover, Neil2-null mice lost significantly more weight compared to WT. Most surprisingly, intrapulmonary delivery of rNEIL2 (14 μg) provided strong protection against RSV infection in both WT and null mice. The inventors then investigated the effect of infection with mouse adaptive strain of SARS-CoV-2, defined as CoV2-MA10 WT and Neil2-null animals. Surprisingly, 80% of CoV2-MA10 infected Neil2^{-/-} mice died within 4-5 days and by contrast, Neil2^{+/+} mice only lost 10-15% of body weight. Moreover, transduction of recombinant (r) NEIL2, but not rNEIL1, blocks SARS-CoV-2's replication in A549-ACE2 cells. The inventors concluded that the individuals with constitutively low level of NEIL2 are at a higher risk of severe illness due to SARS-CoV-2 infection; however, supplementation of NEIL2 will ameliorate such infection.

### I. NEIL2 PEPTIDES

In certain aspects Nei-like protein 2 (NEIL2) or variant thereof has an amino acid sequence that is at least 90, 92, 94, 96, 98, 99, to 100% identical, including all values and ranges there between, over 50, 60, 70, 80, 90, 100, 110, 120, 130, 140, 150, or 155 contiguous amino acids, including all values and ranges there between to MPEGPLVRKFHHLVSPFVGQQVVKTGGSSKKLQPASLQSLWLQDTQVHGKKLFLRFDL DEEMGPPGSSPTPEPPQKEVQKEGAADPKQVGEPSGQKTLDGSSRSAELVPQGEDDSEY LERDAPAGDAGRWLRVSFGLFGSVWVNDFSRAKKANKRGDWRDPSPRLVLHFGGGGF LAFYNCQLSWSSSPVVTPTCDILSEKFHRGQALEALGQAQPVCYTLLDQRYFSGLGNIIK NEALYRAGIHPLSLGSVLSASRREVLVDHVVEFSTAWLQGKFQGRPQHTQVYQKEQCP AGHQVMKEAFGPEDGLQRLTWWCPQCQPQLSEEPEQCQFS (Accession AAH13952.1 NEIL2 protein [Homo sapiens]) (SEQ ID NO:1). Other aspects are directed to an NEIL2 polypeptide, segment, or variant thereof having 10, 20, 30, 40, 50, 60, 70, 80, 90, 100, 110, 120, 130, 140, 150, or 155 contiguous amino acids (including all values and ranges there between) starting from or ending at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, or 332 of SEQ ID NO:1. The NEIL2 polypeptide, segment or variant at least, at most, or about 80, 85, 90, 92, 94, 69, 98, 99, to 100% identical to SEQ ID NO:1, including all values and ranges there between. Preferably, but not necessarily, the segment or variant is a functional segment or variant maintaining a DNA glycosylase activity. In still further aspects an NEIL2 polypeptide, segment or variant can be modified by chemical modification of amino acid side chains (e.g., crosslinking, glycosylation, etc.) or by including or coupling heterologous peptide sequences or targeting moieties at the amino or carboxy terminus of the peptide.

In certain embodiments a NEIL2 polypeptide, segment, or variant is coupled to a lung cell targeting moiety or a carrier peptide directly or indirectly. In some embodiments, the carrier peptide is selected from KKKKAAVALLPAVLLALLAPMSVLTPLLLRGLTGSARRLPVPRAKIHSL (SEQ ID NO:2); KKKKKKKKAAVALLPAVLLALLAPMSVLTPLLLRGLTGSARRLPVPRAKIHSL (SEQ ID NO:3); KKKKKKKKKKKKAAVALLPAVLLALLAPMSVLTPLLLRGLTGSARRLPVPRAKIHSL (SEQ ID NO: 4); KKKKKKKKKKKKKKKKAAVALLPAVLLALLAPMSVLTPLLLRGLTGSARRLPVPRAKI HSL (SEQ ID NO: 5); KKKKAAVALLPAVLLALLAP (SEQ ID NO: 6); KKKKKKKKAAVALLPAVLLALLAP (SEQ ID NO: 7); KKKKKKKKKKKKAAVALLPAVLLALLAP (SEQ ID NO: 8); KKKKKKKKKKKKKKKKAAVALLPAVLLALLAP (SEQ ID NO: 9); YKKKKKKKKKKKKKKKKAAVALLPAVLLALLAP (SEQ ID NO: 10); KKKKKKKKKKKKKKKKAAVALLPAVLLALLAPAAVALLPAVLLALLAP (SEQ ID NO: 11); AAVALLPAVLLALLAPKKKKKKKKKKKKAAVALLPAVLLALLAP (SEQ ID NO: 12); KKKKKKKKKKKKKKKKAAVWLLWYVLLFLLYL (SEQ ID NO: 13); KKKKKKKKKKKKKKKKFWVWLLWYVLLFLLYL (SEQ ID NO:14). In a particular aspect the carrier peptide is SEQ ID NO:9 (K16SP peptide).

Amino acid sequence variants or derivatives of the proteins, polypeptides and peptides of the present invention can be substitutional, insertional or deletion variants, as well as inclusion of amino acid analogs or derivatives. Deletion variants lack one or more residues of the native protein that are not essential for function or immunogenic activity. Another common type of deletion variant is one lacking secretory signal sequences or signal sequences directing a protein to bind to a particular part of a cell or membrane spanning regions or other functional sequences not needed for the in vivo activity sought. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within a polypeptide or peptide, and may be designed to modulate one or more properties, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” or “functional segment/fragment” is well understood in the art and is further defined in detail herein. Accordingly, a biologically functional equivalent may have a sequence of about 70, 75, 80, 85, 90, 95, 96, 97, 98, 99% of amino acids that are identical or functionally equivalent to the amino acids of a polypeptide or peptide and provide a similar biological activity/response.

In certain embodiments a rNEIL2 polypeptide, segment or variant is coupled, covalently or non-covalently with a carrier peptide. A carrier peptide can include, but is not limited to a peptide comprising one or more of a transport peptide; a peptide cleavage sequence; a nuclear localization sequence; and SP is a signal peptide.

A transport protein can be lysine or a non-natural lysine derivative, arginine or a non-natural arginine derivative, and combinations thereof. In some embodiments, the transport protein can be a sequence of four or more lysine residues or a sequence of 4 or more arginine residues. Non-limiting examples of transport proteins can include KKKK (SEQ ID NO: 15); KKKKKKKK (SEQ ID NO:16); KKKKKKKKKKKK (SEQ ID NO:17); KKKKKKKKKKKKKKKK (SEQ ID NO:18); RRRR (SEQ ID NO: 19); RRRRRRRR (SEQ ID NO: 20); RRRRRRRRRRRR (SEQ ID NO: 21); RRRRRRRRRRRRRRRR (SEQ ID NO: 22); KRKR (SEQ ID NO: 23); KKKR (SEQ ID NO: 24); KKKRRRKKKRRR (SEQ ID NO: 25); and KKKKRRRRKKKKRRRR (SEQ ID NO: 26).

A peptide cleavage sequence (PCS) can be any suitable sequence that once in the cell can be enzymatically cleaved from the remaining sequence. In some embodiments, the PCS can include a furin protease cleavage recognition sequence. In other embodiments, the PCS is XRXLRRX (SEQ ID NO: 27), wherein X is a hydrophobic amino acid (e.g., V, I, L, M, F, W, C, A, Y, H, T, S, P, or G).

A nuclear localization sequence (NLS) can be any sequence suitable for targeting the carrier peptide from the cytoplasm into the nucleus of the cell across the nuclear membrane. Any peptide, derivative thereof, or peptide analogue that functions to transport an associated molecule through a nuclear membrane can be used. Certain preferred specific NLSs include PKKKRKV (SEQ ID NO: 28) which is a monopartite NLS from SV40 large T antigen, LVRKKRKTEEESPLKDKDAKKSKQE (SEQ ID NO:29) which is a bipartite NLS from SV40 N1 protein, and PEVKKKRKPEYP (SEQ ID NO: 30).

A signal peptide (SP) can be any sequence capable of translocating across the cell membrane into the interior of the selected target cell. In some cases, a SP is capable of translocating into the interior of an organelle (e.g., mitochondrion or nucleus). For example, a SP can comprise the sequence: X₁X₂VX₃LLX₄X₅VLLX₆LLX₇X₈ (SEQ ID NO: 31) wherein X₁-X₈ are independently L, A, W, F, Y, or V. In some embodiments, X₁ is A or F; X₂ is A or W; X₃ is A or W; X₄ is P or W; X₅ is A or Y; X₆ is A or F; X₇ is A or Y; X₈ is P or L. In some cases, the SP is AAVALLPAVLLALLAP (SEQ ID NO:32). In another embodiment, the SP is AAVALLPAVLLALLAPMSVLTPLLLRGLTGSARRLPVPRAKIHSL (SEQ ID NO: 33).

The peptides disclosed herein are described using the standard one letter amino acid abbreviations. The amino acids can be in their D or L form. All peptides can be prepared using methods known to those having ordinary skill in the art, including solid phase or recombinant methods.

Further provided herein are complexes comprising a biologically active molecule associated with a carrier peptide. In some embodiments, the biologically active molecule is non-covalently bound to the carrier peptide. A carrier peptide can comprise any sequence as described previously.

“Associated with” or “coupled to” as used herein is meant that the biologically active molecule is conjugated to the carrier peptide in such a manner that when the carrier peptide crosses the cell membrane, the molecule is also imported across the cell membrane. In certain embodiments, the biologically active molecule is non-covalently bound to the carrier peptide. In other embodiments, the carrier peptide may be covalently bound, either directly or indirectly (e.g., through a linker), to the biologically active molecule.

A linker can be any moiety suitable for linking a carrier peptide to a biologically active molecule. A linker can be bound at the C-terminus, the N-terminus, or both, of a carrier peptide. Additionally, a linker can be bound to the side chain of a carrier peptide. If a carrier peptide is bound to multiple linkers, each linker can be different. A linker can be covalently linked to a side chain of an amino acid, e.g., lysine, glutamine, cysteine, methionine, glutamate, aspartate, asparagine.

In some embodiments an amino acid side chain can serve as the linker. For example the epsilon amino group (ε-NH₂) can be used to conjugate to a carrier for instance through an amide or thiourea linkage. Similarly, the delta amino group of ornithine (orn), the gamma amino group of diaminobutyric acid (dab), or the beta amino group of diamino proprionic acid (dpr) can also act as linkers. These amino acids may be at the C- or N-terminus of the carrier peptide or they may be positioned within the carrier peptide sequence.

A. Polypeptide Composition and Formulations

“Polypeptide” refers to any peptide or protein comprising amino acids joined by peptide bonds or modified peptide bonds. “Polypeptide” refers to short chains, including peptides, oligopeptides or oligomers; and to longer chains, including proteins. Polypeptides may contain amino acids other than the 20 gene-encoded amino acids. “Polypeptides” include amino acid sequences modified either by natural processes, such as post-translational processing, or by chemical modification or other synthetic techniques well known in the art. Modifications can occur anywhere in a polypeptide, including the peptide backbone, the amino acid side-chains, and the amino terminus or the carboxy terminus. It will be appreciated that the same type of modification may be present in the same or varying degrees at several sites in a given polypeptide. Also, a given polypeptide may contain many types of modifications. Modifications include terminal fusion (N- and/or C-terminal), acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel.

The term “amino acid” or “residue” should be understood to mean a compound containing an amino group (NH₂), a carboxylic acid group (COOH), and any of various side groups, that have the basic formula NH₂CHRCOOH, and that link together by peptide bonds to form proteins. Amino acids may, for example, be acidic, basic, aromatic, polar or derivatized. A one-letter abbreviation system is frequently applied to designate the identities of the twenty “canonical” amino acid residues generally incorporated into naturally occurring peptides and proteins, these designation are well known in the art. Such one-letter abbreviations are entirely interchangeable in meaning with three-letter abbreviations, or non-abbreviated amino acid names. The canonical amino acids and their three letter and one letter codes include Alanine (Ala) A, Glutamine (Gln) Q, Leucine (Leu) L, Serine (Ser) S, Arginine (Arg)R, Glutamic Acid (Glu) E, Lysine (Lys) K, Threonine (Thr) T, Asparagine (Asn) N, Glycine (Gly) G, Methionine (Met) M, Tryptophan (Trp) W, Aspartic Acid (Asp) D, Histidine (His) H, Phenylalanine (Phe) F, Tyrosine (Tyr) Y, Cysteine (Cys) C, Isoleucine (Ile) I, Proline (Pro) P, and Valine (Val) V.

Certain embodiments also include variants of the polypeptides described herein. Variants of the disclosed polypeptides may be generated by making amino acid additions or insertions, amino acid deletions, amino acid substitutions, and/or chemical derivatives of amino acid residues within the polypeptide sequence. Desired amino acid substitutions (whether conservative or non-conservative) can be determined by those skilled in the art in accordance with guidance provided herein for increasing stability, while maintaining or enhancing potency of the polypeptides as determined for example the assays and procedures described herein. In certain embodiments, conservative amino acid substitutions can encompass non-naturally occurring amino acid residues which are typically incorporated by chemical peptide synthesis rather than by synthesis in biological systems.

Conservative modifications can produce peptides having functional, physical, and chemical characteristics similar to those of the peptide from which such modifications are made. In contrast, substantial modifications in the functional and/or chemical characteristics of peptides may be accomplished by selecting substitutions in the amino acid sequence that differ significantly in their effect on maintaining (a) the structure of the molecular backbone in the region of the substitution, for example, as an α-helical conformation, (b) the charge or hydrophobicity of the molecule at the target site, or (c) the size of the molecule. For example, a “conservative amino acid substitution” may involve a substitution of a native amino acid residue with a non-native residue such that there is little or no effect on the polarity or charge of the amino acid residue at that position.

Recombinant DNA- and/or RNA-mediated protein expression and protein engineering techniques, or any other methods of preparing peptides, are applicable to the making of the polypeptides disclosed herein or expressing the polypeptides disclosed herein in a target cell or tissue. The term “recombinant” means that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. Examples of such molecular biological procedures are found in Maniatis et al., Molecular Cloning. A Laboratory Manual, Cold Spring Harbor Laboratory, Cold Spring Harbor, N.Y., 1982. A “recombinant DNA molecule,” is comprised of segments of DNA joined together by means of such molecular biological techniques. The term “recombinant protein” or “recombinant polypeptide” as used herein refers to a protein molecule that is expressed using a recombinant DNA molecule. A “recombinant host cell” is a cell that contains and/or expresses a recombinant nucleic acid.

The polypeptides can be made in transformed host cells according to methods known to those of skill in the art. Briefly, a recombinant DNA molecule, or construct, coding for the peptide is prepared. Methods of preparing such DNA molecules are well known in the art. The polypeptides can also be made by synthetic methods. Solid phase synthesis can be used as a technique of making individual polypeptides since it is the most cost-effective method of making small peptides.

A composition that includes a polypeptide covalently linked, attached, or bound, either directly or indirectly through a linker moiety, to another peptide, vehicle (e.g., carrier), or a half-life extending moiety is a “conjugate” or “conjugated” molecule, whether conjugated by chemical means (e.g., post-translationally or post-synthetically) or by recombinant fusion. Conjugation of the polypeptides can be via the N-terminus and/or C-terminus of the polypeptide, or can be intercalary as to the peptide's primary amino acid sequence. A linker can be used to create fusion protein(s) that allow introduction of additional moieties to enhance uptake or localization of a polypeptide.

In some embodiments, a polypeptide is coupled to or encapsulated in a delivery vehicle, such as a carrier (e.g., a particle), or a liposome. In some embodiments, coupling of the polypeptide to the carrier includes one or more covalent and/or non-covalent interactions. In one embodiment the carrier is a metallic or polymeric particle. In one embodiment, the carrier is a liposome. The particles can be microscopic or nanoscopic in size. In certain aspects a particle has a diameter of from at least, at most, or about 0.1 μm to at least, at most, or about 10 μm. In another aspect, the particle has an average diameter of at least, at most, or about 0.3 μm to at least, at most, or about 5 μm, 0.5 μm to at least, at most, or about 3 μm, or 0.2 μm to at least, at most, or about 2 μm. In certain aspects the particle can have an average diameter of at least, at most, or about 0.1 μm, or at least, at most, or about 0.2 μm or at least, at most, or about 0.3 μm or at least, at most, or about 0.4 μm or at least, at most, or about 0.5 μm or at least, at most, or about 1.0 μm or at least, at most, or about 1.5 μm or at least, at most, or about 2.0 μm or at least, at most, or about 2.5 μm or at least, at most, or about 3.0 μm or at least, at most, or about 3.5 μm or at least, at most, or about 4.0 μm or at least, at most, or about 4.5 μm or at least, at most, or about 5.0 μm, including all values and ranges there between.

In some embodiments, the charge of a carrier (e.g., positive, negative, neutral) is selected to impart application-specific benefits (e.g., physiological compatibility, beneficial surface-peptide interactions, etc.). In some embodiments, a carrier has a net neutral or negative charge (e.g., to reduce non-specific binding to cell surfaces which, in general, bear a net negative charge). In some instances, a carrier is coupled to multiple polypeptides and can have 2, 3, 4, 5, 6, 7, 8, 9, 10 . . . 20 . . . 50 . . . 100, or more copies of a certain polypeptide or combinations of polypeptides exposed on the surface. In some embodiments, a carrier displays a single type of polypeptide. The terms “packaged”, “encapsulation” and “entrapped,” as used herein, refer to the incorporation or association of a polypeptide in or with a liposome or similar vehicle. The polypeptide may be associated with the lipid bilayer or present in the aqueous interior of the liposome, or both.

B. Expression and Expression Vectors

Polypeptide(s)/peptide(s) described herein can be encoded by a nucleic acid that can in turn be inserted into or employed with a suitable expression vector or system. Recombinant expression can be accomplished using a vector, such as a plasmid, virus, etc. The vector can include a promoter operably linked to nucleic acid encoding one or more polypeptides. The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing nucleic acids encoding proteases can be produced. Such expression vectors include, for example, pET, pET3d, pCR2.1, pBAD, pUC, and yeast vectors. The vectors can be used, for example, in a variety of in vivo and in vitro situations. The vector may be a gene therapy vector, for example an adenovirus vector, a lentivirus vector or a CRISP-R vector.

The expression cassette, expression vector, and sequences in the cassette or vector can be heterologous to a particular nucleic acid or cell. As used herein, The term “heterologous” when used with respect to a nucleic acid (DNA or RNA) or protein refers to a nucleic acid or protein (also named polypeptide or enzyme) that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are typically not endogenous to the cell into which it is introduced, but has been obtained from another cell or synthetically or recombinantly produced. An expression cassette, expression vector, regulatory sequence, promoter, or nucleic acid can refer to an expression cassette, expression vector, regulatory sequence, or nucleic acid that has been manipulated in some way. For example, a heterologous promoter can be a promoter that is not naturally linked to a nucleic acid to be expressed, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that are linked to a coding region to which they are not linked in nature.

Another aspect of the invention is directed to a gene therapy vector comprising a rNEIL2 gene construct. Gene therapy vectors are known in the art and include, but are not limited to, lentiviral vectors, adenoviral vectors, adeno-associated viral vectors, plasmids and the like. Construction of a gene therapy vector of the invention can be done by methods known in the art. In certain aspects a gene therapy vector can be administered in an amount of about, at most, or at least 10, 100, 1000, 1×10⁴, 1×10⁵, 1×10⁶, 1×10⁷, 1×10⁸, 1×10⁹, 1×10¹⁰, 1×10¹¹, 1×10¹² viral particles (VP) or colony forming units (CFU), including all values and ranges there between. As an example of a gene therapy vector a rNEIL expression cassette can be included in a lentiviral vector. The therapeutic vector can be transduced into cells ex vivo and the cells delivered to the patient or subject. Likewise, a therapeutic vector of the invention can be delivered directly to the patient.

### II. IMMUNE DYSREGULATION

In one aspect immune dysregulation can include a cytokine storm (hypercytokinemia), cytokine storm is the systemic expression of a healthy and vigorous immune system resulting in the release of more than 150 inflammatory mediators (cytokines, oxygen free radicals, and coagulation factors). Both Th1 pro-inflammatory cytokines and Th2 anti-inflammatory cytokines are elevated in the serum of patients experiencing a cytokine storm. Cytokine storms potentially damage body tissues and organs, may result in death, and can occur in a number of infectious diseases including but not limited to COVID-19, avian influenza, and swine influenza.

Certain embodiments are directed to methods for the treatment of a viral pathogen (e.g., SARS-CoV-2) infection in a subject in need of such treatment. The methods can include the step of administering to a subject a therapeutically effective amount of at least one isolated NEIL2 peptide as defined herein, or of a composition comprising the at least one isolated NEIL2 peptide. This treatment can control and inhibit immune dysfunction emanating from viral pathogen infection. In certain aspects the method of treatment inhibits the progression of said viral infection or the pathogenesis of the viral infection, preferably reducing the morbidity rate and/or mortality rate in subjects infected with the virus. In certain instances the methods of treatment prevent worsening, arrests and/or ameliorates at least one symptom of the viral infection or damage to said subject or an organ (e.g., lungs) or tissue of said subject, emanating from or associated with said viral infection. The symptom of the viral infection or said damage can be at least one of fever (temperature of >38° C.), acute respiratory distress syndrome (ARDS), multiple organ dysfunction syndrome (MODS), systemic inflammatory response syndrome (SIRS), hypotension, tachycardia, dyspnea, ischemia, insufficient tissue perfusion (especially involving the major organs), uncontrollable hemorrhage, multisystem organ failure primarily due to hypoxia or tissue acidosis), and/or severe metabolism dysregulation.

According to a second aspect, disclosed herein are methods for the treatment of a viral pathogen infection in a subject in need of such treatment, comprising administering to a subject a therapeutically effective amount of at least one isolated NEIL2 peptide as defined herein, or of a composition comprising said at least one isolated NEIL2 peptide, and another, additional antiviral therapeutic agent.

By the term “additional antiviral therapeutic agent” (or “drug”) as used herein is meant that the additional antiviral therapeutic agent/drug (which per se exhibits antiviral activity) is other than the peptide/s as defined herein.

Generally, the term “viruses” is used in its broadest sense to include coronaviruses (SARS, SARS-CoV-2, MERS etc.), flaviviruses, haemorrhagic fever viruses such as West Nile virus and ZIKA virus, adenoviruses, papovaviruses, herpesviruses, CMV, pox viruses; hepatitis A, hepatitis B, hepatitis C, rhinoviruses, rubella virus, arboviruses, Orthomyxoviridae viruses such as influenza viruses A and B; measles virus, mumps virus, Filoviridae members such as Ebola virus and Marburg virus, virus as well as others, as described in more detail below.

In some embodiments of aspects of the present disclosure, the viral pathogen is a virus of any one of the Coronaviridae, Pneumoviridae, Orthomyxoviridae, Filoviridae, Flaviviridae, or Poxviridae families, and subfamilies thereof. In certain aspects a viral pathogen infection may be diagnosed by a physician, i.e., the subject has a viral infection.

Methods, compositions, combinations, and kits according to the present disclosure provide for the treatment of viral infection by a large number of viruses. These include, but are not limited to, the following specific viruses, described in detail.

In certain embodiments the viral pathogen is a Coronaviridae family member, more particularly the Severe Acute Respiratory Syndrome (SARS) virus (SARS-CoV or SARS-CoV-2), causing a viral respiratory disease of zoonotic origin. Initial symptoms are flu-like and may include fever, muscle pain, lethargy symptoms, cough, sore throat, and other nonspecific symptoms. The only symptom common to all patients appears to be a fever above 38° C. (100° F.). SARS may eventually lead to shortness of breath and/or pneumonia; either direct viral pneumonia or secondary bacterial pneumonia.

The term “Coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2)N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (GenBank accession number NC 045512.2), coV (ATCC accession #VR-1475), human coV 229E (ATCC accession #VR-740), human coV OC43 (ATCC accession #VR-920), and SARS-coronavirus (Center for Disease Control).

Pneumoviridae is a family of negative-strand RNA viruses in the order Mononegavirales. Humans, cattle, and rodents serve as natural hosts. Respiratory tract infections are associated with member viruses such as human respiratory syncytial virus (RSV). There are five species in the family which are divided between the genera Metapneumovirus (including human metapneumovirus (hMPV) and Orthopneumovirus (including human respiratory syncytial virus (hRSV).

Influenza A and B virus particles contain a genome of negative sense, single-strand RNA divided into 8 linear segments. Co-infection of a single host with two different influenza viruses may result in the generation of reassortant progeny viruses having a new combination of genome segments, derived from each of the parental viruses. Type A influenza viruses are divided into subtypes based on two proteins on the surface of the virus, hemagglutinin (HA) and neuraminidase (NA). There are 15 different HA subtypes and 9 different NA subtypes. Subtypes of influenza A virus are named according to their HA and NA surface proteins. For example, an “H7N2 virus” designates influenza A subtype that has an HA 7 protein and an NA 2 protein, etc. All known subtypes of A viruses can be found in birds. Symptoms of human infection with avian viruses have ranged from typical flu-like symptoms (fever, cough, sore throat and muscle aches) to eye infections, pneumonia, severe respiratory diseases (such as acute respiratory distress), and other severe and life-threatening complications. The symptoms of bird flu may depend on which virus caused the infection. Each of avian influenza A viruses H5, H7, and H9 theoretically can be partnered with any one of nine neuraminidase surface proteins; thus, there are potentially nine different forms of each subtype (e.g., H5N1 to H5N9). H5 infections have been documented in humans, sometimes causing severe illness and death. H7 infection in humans is rare, but can occur among persons who have direct contact with infected birds. In certain aspects the viral pathogen is avian Influenza virus type A virus, or any subtype and reassortant thereof. In other particular embodiments of all aspects of the present disclosure, the viral pathogen is avian Influenza type A virus has haemagglutinin component of subtype H5, H7 or H9. In some particular aspects the viral pathogen is swine Influenza type A virus subtype H1N1. In further specific embodiments of all aspects of the present disclosure, the viral pathogen is avian Influenza type A virus subtype H5N1.

In further aspects and embodiments of the present disclosure, the viral pathogen can be a virus belonging to the filoviridea family, also referred to herein as “Filoviruses”. These are generally single-stranded negative sense RNA viruses that typically infect primates. Filoviruses are able to multiply in virtually all cell types. The filovirus genome comprises seven genes that encode 4 virion structural proteins (VP30, VP35, nucleoprotein, and a polymerase protein (L-pol)) and 3 membrane-associated proteins (VP40, glycoprotein (GP), and VP24). Filoviruses cause hemorrhagic fevers with high levels of fatality. They are classified in two genera within the family Filoviridae: Ebola virus (EBOV) and Marburg virus (MARV), both being highly pathogenic in humans and nonhuman primates, with case fatality levels of up to 90%. Ebola virus species Reston (REBOV) is pathogenic in monkeys but does not cause disease in humans or great apes. Fatal outcome in filoviral infection is associated with an early reduction in the number of circulating T cells, failure to develop specific humoral immunity, and the release of pro-inflammatory cytokines. More specifically, these viruses cause sporadic epidemics of human disease characterized by systemic hemorrhage, multi-organ failure and death in most instances. The onset of illness is abrupt, and initial symptoms resemble those of an influenza-like syndrome. Fever, headache, general malaise, myalgia, joint pain, and sore throat are commonly followed by diarrhea and abdominal pain.

In certain aspects the viral pathogen can be a virus belonging to the Flaviviridea family, also referred to herein as “Flaviviruses”, specifically West Nile virus (WNV), Dengue virus (DENV), Yellow Fever virus (YFV) or the Zika virus (ZIKV), which are usually mosquito-borne. WNV causes West Nile Fever, which can be manifested by fever, headache, vomiting, or a rash. Encephalitis or meningitis are rather rare. Recovery may take weeks to months. DNV is the cause for Dengue fever, with symptoms typically beginning three to fourteen days after infection, which may include a high fever, headache, vomiting, muscle and joint pains, and a characteristic skin rash. Recovery generally takes two to seven days. In a small proportion of cases, the disease develops into the life-threatening dengue hemorrhagic fever, resulting in bleeding, low levels of blood platelets and blood plasma leakage, or into dengue shock syndrome, where dangerously low blood pressure occurs. YFV causes Yellow Fever, viral disease of typically short duration. In most cases, symptoms include fever, chills, loss of appetite, nausea, muscle pains particularly in the back, and headaches. Symptoms typically improve within five days. In about 15% of people, within a day of improving the fever comes back, abdominal pain occurs, and liver damage begins causing yellow skin. If this occurs, the risk of bleeding and kidney problems is also increased. ZKV causes a self-limiting, dengue fever (DF)-like disease with an incubation time of up to 10 days. Signs and symptoms consist of rather low-grade fever, myalgia and a maculopapular rash, accompanied by arthralgia and headache, and less often edema, sore throat, and vomiting.

According to aspects and embodiments of the present disclosure, where an additional antiviral therapeutic agent is used, such agent may be, for example but not limited to, a viral neuraminidase inhibitor (for example Oseltamivir or Zanamivir), a viral polymerase inhibitor (for example Ribavirin) or M2 ion-channel blocker (for example amantadine or rimantadine).

In certain embodiments the additional antiviral agent is administered in a therapeutically effective amount (also referred herein “therapeutic dose”). A therapeutically “effective amount” or “therapeutic dose” for purposes herein is that determined by such considerations as are known in the art. The amount must be sufficient to inhibit, prevent worsening of, arrest and/or ameliorate at least of one symptom of the said viral infection in the treated subject, and/or prevent damage to said subject or an organ or tissue of the subject emanating from or associated with said viral infection by the infecting virus, which may lead to cytokine storm. The said at least one symptom of the said viral infection or damage to said subject or an organ or tissue of the subject, emanating from or associated with said viral infection is at least one of fever (temperature of >38° C.), acute respiratory distress syndrome (ARDS), multiple organ dysfunction syndrome (MODS), systemic inflammatory response syndrome (SIRS), hypotension, tachycardia, dyspnea, ischemia, insufficient tissue perfusion (especially involving the major organs), uncontrollable hemorrhage, multisystem organ failure primarily due to hypoxia or tissue acidosis), severe metabolism dysregulation.

The terms “treat”, or forms thereof, and also the terms “inhibit”, “arrest”, and “ameliorate” and forms thereof mean to at least partially cure the patient's disease or condition.

By the term “achieving a therapeutic effect” it is meant, for example, slowing down or preventing the progression of viral infection symptoms, preventing worsening, arresting and/or ameliorating at least one of the viral infection symptoms, preventing damage to the treated subject or to an organ or tissue of said subject, emanating from or associated with said viral infection, and preventing death of the subject.

Treatment with any of the compositions, combined compositions or kits of the present disclosure may increase survival of the treated subjects by at least 10%, at least 15%, at least 20%, at least 25%, at least 30%, at least 40%, at least 50%, at least 60%, at least 70%, at least 80% or even by at least 90% or 100% as compared to the survival of untreated subjects.

It is further noted that treatment with any of the compositions, combined compositions and kits of the invention may improve any measured parameter for lung function, for example, the oxygen saturation, that may increase by at least 5%, at least 10%, at least 15%, at least 20%, at least 25%, at least 30%, at least 40%, at least 50% or even at least 80%, 90% or 100% as compared to the level prior to treatment. Similar improvement may be also shown in parameters such as body weight, and lung lobe histology.

By the term “a subject in need of such treatment” as referred to herein it is meant a subject (human, animal) diagnosed as inflicted with a viral pathogen infection by a skilled physician. The symptoms of the viral infection or damage to said subject or an organ or tissue of said subject, emanating from or associated with said viral infection are well known to a skilled physician.

Although the methods, peptides, uses, compositions, combinations and kits of the present disclosure are particularly intended for the treatment viral infection in mammals, particularly humans, other mammals, and also avian and particularly domestic birds are included. Domestic birds may be but are not limited to chicken, turkeys, geese, ducks, pheasants, quails, pigeons and ostriches. By way of non-limiting examples, mammalian subjects also include monkeys, equines, cattle, canines, felines, rodents such as mice and rats, and pigs.

Therefore in the above and other embodiments the subject is a human subject. In other particular embodiments the subject is an avian (a bird).

### III. PHARMACEUTICAL FORMULATIONS AND ADMINISTRATION

Certain embodiments include compositions including rNEIL2 or a variant thereof with one or more of the following: a pharmaceutically acceptable diluent; a carrier; a solubilizer; an emulsifier; and/or a preservative. Thus, the use of one or more rNEIL2 agent described herein in the preparation of a pharmaceutical composition of a medicament is also included. Such compositions can be used in the treatment of allergic asthma, allergic rhinitis, or bacterial infections.

The therapeutic agent(s) may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the therapeutic agent(s) that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from at least, at most, or about 4.0 to at least, at most, or about 8.5, or alternatively, between at least, at most, or about 5.0 to 8.0, including all values and ranges there between. Pharmaceutical compositions can comprise TRIS buffer of at least, at most, or about a pH of 6.5-8.5, including all values and ranges there between, or acetate buffer of at least, at most, or about a pH of 4.0-5.5, including all values and ranges there between, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and/or reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, inhalation, instillation, intravenous, intraperitoneal, oral, intralymphatic, subcutaneous administration, intraarterial, intramuscular, intrapleural, intrathecal, and by perfusion through a regional catheter.

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258, 6,794,357, 6,737,045, and 6,488,953, all of which are incorporated by reference. According to certain aspects, at least one therapeutic composition can be delivered by any of a variety of inhalation or nasal devices known in the art for administration of a therapeutic agent by inhalation. Other devices suitable for directing pulmonary or nasal administration are also known in the art. Typically, for pulmonary administration, at least one therapeutic composition is delivered in a particle size effective for reaching the lower airways of the lung or sinuses. Some specific examples of commercially available inhalation devices suitable for the practice of this invention are Turbohaler™ (Astra), Rotahaler® (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler® powder inhaler (Fisons), or the like.

All such inhalation devices can be used for the administration of a therapeutic composition in an aerosol. Such aerosols may comprise either solutions (both aqueous and non-aqueous) or solid particles. Metered dose inhalers typically use a propellant gas and require actuation during inspiration. Dry powder inhalers use breath-actuation of a mixed powder. Nebulizers produce aerosols from solutions, while metered dose inhalers, dry powder inhalers, and the like generate small particle aerosols. Suitable formulations for administration include, but are not limited to nasal spray or nasal drops, and may include aqueous or oily solutions of a therapeutic composition described herein.

Once the therapeutic compositions have been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, at least, at most, or about 0.1% to at least, at most, or about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the therapeutic compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental or pulmonary administration are also sterile, substantially isotonic and made under GMP conditions.

For the compounds described herein, alone or as part of a pharmaceutical composition, such doses are between at least, at most, or about 0.001 mg/kg and 10 mg/kg body weight, preferably between at least, at most, or about 1 and 5 mg/kg body weight, most preferably between 0.5 and 1 mg/kg body weight, including all values and ranges there between.

Therapeutically effective doses will be easily determined by one of skill in the art and will depend on the severity and course of the disease, the patient's health and response to treatment, the patient's age, weight, height, sex, previous medical history and the judgment of the treating physician.

Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

### IV. KITS

Certain embodiments are directed to kits comprising the presently disclosed active compounds and/or compositions thereof. The kit in accordance with the present disclosure can include one or more pharmaceutically active compounds, namely a peptide as disclosed herein and/or an additional antiviral agent. Each of the active compounds, namely a NEIL2 peptide and/or an additional antiviral agent, or compositions comprising the same, can be comprised in a separate unit dosage form, the kit thus containing two individual first and second unit dosage forms, respectively. The kit can include container means for containing active compounds, the peptide and/or the additional antiviral agent, and/or compositions thereof, such as a divided bottle or a divided foil packet. However, the separate active compounds or compositions thereof can also be contained within a single, undivided container. The separate active compounds, namely the peptide and the additional antiviral agent, or compositions thereof, can also be comprised in a single composition, optionally further comprising a pharmaceutically acceptable additive, carrier or diluent, where the at least one peptide and additional antiviral agent are chemically and pharmacologically compatible, for example, where there is no drug-drug interaction between them. The kit can include reagents for measuring or determining the levels of NEIL2 in a sample.

In other words the present disclosure further provides a kit for the treatment of a viral pathogen infection and/or at least one symptom thereof in a human subject in need of such treatment, comprising: (a) at least one isolated NEIL2 peptide, optionally comprised in a composition further comprising a pharmaceutically acceptable carrier or diluent, optionally in a first dosage unit form; (b) an additional antiviral therapeutic agent, optionally comprised in a composition further comprising a pharmaceutically acceptable carrier or diluent, optionally in a second dosage unit form; (c) container means for containing said first and/or second dosage forms jointly or separately; (d) instructions for use; and optionally (e) device for administering the at least one isolated peptide and/or the at least one additional antiviral therapeutic agent to said subject.

Typically the disclosed kit includes directions for the administration of the separate or combined active compounds. The kit form is particularly advantageous when the separate active compounds are administered in different dosage forms (e.g., oral and injectable, for example intravenous or intraperitoneal), are administered at different dosage intervals, or when titration of the individual components of the combination is desired by the prescribing physician. The kit of the present disclosure can optionally further comprise means for administering the different active compounds, the peptides of the invention and/or the additional antiviral therapeutic agent, or compositions thereof.

According to one embodiment, the disclosed kit is intended for achieving a therapeutic effect in a subject suffering from an infection caused by a viral pathogen, or at least one of the symptoms thereof, as described herein. In other words, in specific embodiments the kit of the present disclosure is for use in the treatment of a viral pathogen infection in a subject in need of such treatment.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

It is now widely accepted that inflammation underlies a wide variety of physiological and pathological processes, including disease caused by viral infections. Reactive oxygen species (ROS) generated due to dysregulated inflammatory response are known to induce significant amount of DNA damage. Most of these DNA lesions are mutagenic and/or cytotoxic and have been implicated in a wide variety of pathophysiological states. The inventors analyzed the expression of DNA repair genes in publicly available transcriptomic datasets and found that the level of NEIL2, an oxidized base specific mammalian DNA glycosylase, is significantly lower in patients who suffered from severe COVID-19 compared to the patients with milder disease. The inventors then analyzed the correlation between NEIL2's level and other aspects of COVID-19 severity, such as requirements for ICU and mechanical ventilators (MV) among the cohorts of COVID-19 and non-COVID-19 patients. Unlike the levels of other DNA glycosylases, patients requiring ICU or MV had significantly lower levels of NEIL2 compared to non-ICU or non-MV patients. Moreover, a study involving 100 hospitalized COVID-19 patients showed a significant correlation between higher NEIL2 levels and shorter duration of hospitalization. Collectively these observations suggest a link between NEIL2 deficiency and COVID-19 severity. Of note, non-COVID patients with lower level of NEIL2 are also severely sick and they were admitted to ICU or needed mechanical ventilation, suggesting NEIL2's overall protective role.

### Example 1

### NEIL2 Expression Level can Serve as a Biomarker for the Severity of SARS-CoV-2 Infection and Disease in Humans

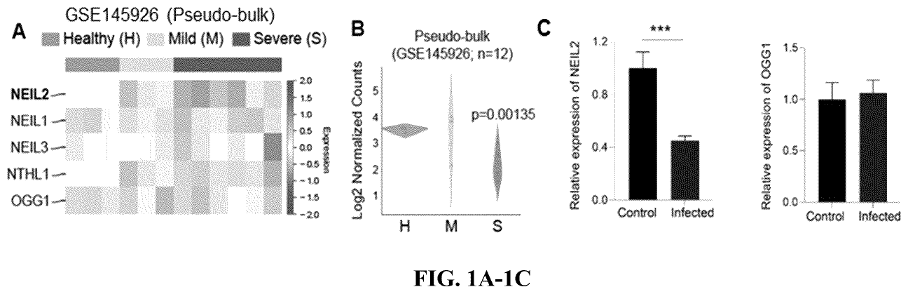

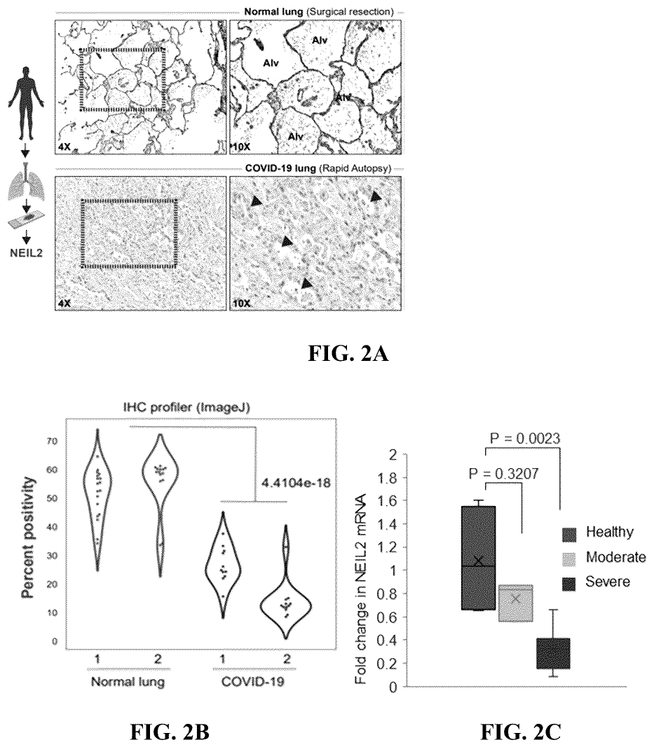

The level of NEIL2 is low in severe COVID-19 patients and SARS-CoV-2 infected permissive animals. SARS-CoV-2 infection-induced release of soluble inflammatory mediators can modulate pulmonary endothelial permeability and influx of inflammatory cells (macrophages, neutrophils, and T cells) to the site of infection leading to uncontrolled inflammation, impairing lung function^{7,18,21-23}. Such host responses also generate reactive oxygen species (ROS) that are not only signal transducers but are also inducers of host genome damage, thereby triggering DNA damage response. However, in the absence of any report on mechanistic link between SARS-CoV-2 infection and the host genome repair, RNA-seq data was analyzed, available in public database (GSE 145926), and several other GSEs (GSE150728, GSE152641 and GSE161777) of SARS-CoV-2 infected patients. Surprisingly, the level of DNA glycosylase NEIL2, among other DNA repair proteins (˜100), found to be significantly lower in severe COVID-19 patients relative to the control population (FIGS. 1A, 1B). Decreased level of NEIL2, but not of 8-oxoguanine DNA glycosylase (OGG1), another oxidized DNA base repair enzyme, was further confirmed by qRT-PCR analysis of SARS-CoV-2 infected hamster lung (FIG. 1C). Immunohistochemical (IHC) analysis of lung autopsy of COVID-19 patients (FIGS. 2A, 2B) also showed significant decrease in the NEIL2 level, particularly in alveolar epithelial cells.

Peripheral blood mononuclear cells (PBMCs) serve as an excellent alternative to the costly and challenging approach of obtaining airways samples from severe COVID-19 patients. Therefore, NEIL2 transcript level was analyzed in PBMCs isolated from COVID-19 patients admitted to the University of Texas Medical Branch (UTMB) hospital and found that the NEIL2 level was significantly lower in severe COVID-19 patients compared to that in patients with milder symptoms (FIG. 2C). These results strongly support a correlation between NEIL2 level in PBMC and the severity of SARS-CoV-2 infection.

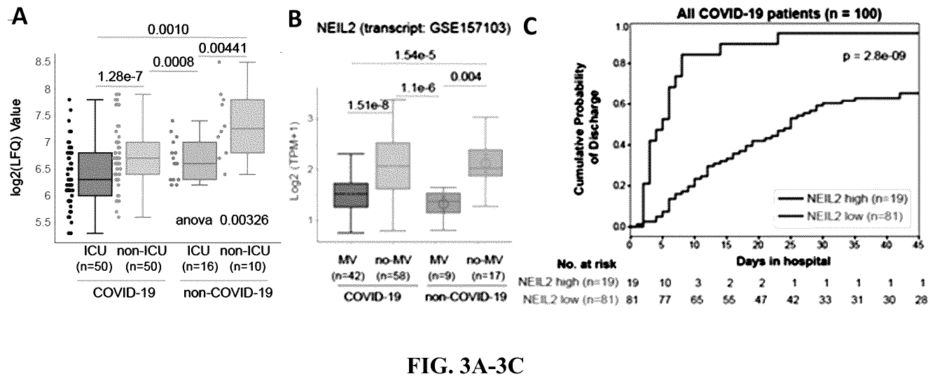

The correlation between NEIL2 expression and the requirement of ICU and mechanical ventilators (MV) was examined among the cohorts of COVID-19 and non-COVID-19 patients. Unlike the insignificant difference observed in the levels of other DNA glycosylases (e.g., OGG1 and NEIL3), patients requiring ICU or under MV had significantly lower levels of NEIL2 compared to non-ICU or non-MV patients (FIGS. 3A, 3B). Moreover, a study involving 100 hospitalized COVID-19 patients showed a significant correlation between higher NEIL2 levels and shorter duration of hospitalization (FIG. 3C). Collectively, these observations support a strong link between NEIL2 deficiency and COVID-19 severity.

Based on the preliminary studies, the following studies are to be performed:

(a) Confirmation of the correlation between the level of NEIL2 expression, disease severity, and duration of the hospital stay. Compromised DNA repair capacity of the individuals could play a critical role in disease severity. Baseline endogenous DNA damage in PBMCs can serve as prognostic marker for identifying such individual^{24,25}. One goal is to confirm that NEIL2 expression level in the PBMCs can be used a reliable biomarker for the severity of SARS-CoV-2 infection. UTMB routinely treats many COVID-19 patients. The Biorepository unit is continuously collecting plasma/sera and banking blood samples of COVID-19 patients, as well as of healthy individuals. The inventors will analyze expression level of DNA repair genes by using RT² profiler PCR array (Qiagen) from PBMCs of at least 250 patients and equal numbers of non-covid controls. Validation of NEIL2 expression and other repair proteins for oxidized DNA bases (including OGG1, NEILL NEIL3, NTH1 and MYH1, as controls) are done by gene-specific qRT-PCR at the transcript level and also at the protein level by Western analysis in the lysates derived from of patients PBMCs.

Examination of whether level of NEIL2 in PBMCs reflects its expressions in lungs, is performed by IHC analysis of NEIL2 (OGG1, NEIL1 as control) of postmortem patients' lung autopsies minimally from 20 COVID-19 and non-covid controls. Analysis by quantitative confocal microscopy in bronchiolar and alveolar epithelial cells, the site of viral replication and primary source of inflammatory mediators, will further validate NEIL2's key role in COVID-19 pathogenesis.

In view of the mounting evidence for hyperinflammation as the primary cause of deteriorating lung function in COVID-19 patients and characterization of candidate inflammatory cytokines triggering such inflammation²⁶, inflammatory profiling is conducted from sera using Bio-Plex Multiplex Immunoassays (Bio-Rad). All data will be analyzed and correlated with the severity of disease and the duration of hospital stay. Together, these data will help establish the usefulness of the level of NEIL2 expression as a prognostic biomarker for disease severity. This could be a powerful tool for the attending physicians for treating COVID-19 patients, as it would help to improve the prognosis, based on the prediction of disease severity.

(b) Analysis of DNA damage in COVID-19 patients. Based on accumulating evidence that SARS-CoV-2 infection induce generation of ROS, and also inhibits DNA repair^{27,28}, studies will quantify the overall impact of infection on host genome damage. Genomic DNA is isolated from the PBMCs of COVID-19 patients (mild and severe) as well as healthy individuals and gene-specific long amplicon qPCR (LA-qPCR) will be performed as described previously¹⁷. Several reports indicated that COVID-19 patients accumulate dysfunctional mitochondria leading to abnormal ROS production and altered mitochondrial physiology²⁹⁻³¹. NEIL2 also plays an important role in mitochondrial genome maintenance³². Mitochondrial genome damage is analyzed from such samples following established methods³². Collectively, these studies will generate a comprehensive view of host genome damage induced by CoV-2 infection.

(c) Testing SNP (rs804270) located at the promoter region of NEIL2 for correlation with its expression level and disease severity. A recent report indicated that the NEIL2 SNP rs804270 located in the regulatory region of its own promoter, significantly affects both transcript and protein level³³. Studies will test if this SNP is a significant risk factor for susceptibility to SARS-CoV-2 infection-induced COVID-19. The frequencies of the GG (WT), GC and CC genotypes of rs804270 will be identified in the PBMCs isolated from the same non-covid control and subjects (˜250). The variant will be identified and characterized in COVID-19 patients using allele-specific qPCR, as was previously carried out^{34,35}. In parallel, the NEIL2 transcript will be quantified using high throughput q-RTPCR. These studies will verify if this SNP is etiologically linked to the expression of pro-inflammatory cytokines by measuring the protein (from sera) and RNA (from PBMC). Establishing association of host genetic variants with severity of C(I) ID-19 induced morbidity could help identify possible therapeutic targets.

Expected results and data interpretation. The inventors will document that lower NEIL2 expression in hospitalized inpatients with severe COVID-19 infection relative to that in COVID-19 free individuals is statistically significant and correlates with exacerbated inflammatory response. Furthermore, it will be confirmed that the population group carrying the NEIL2 SNP rs804270 has lower NEIL2 level, more DNA damage and is more vulnerable to CoV-2 infection. It is contemplated that the individuals with low baseline DNA damage are likely to be more resilient to viral infection. So, data derived from these studies will allow further confirmation the role of NEIL2 in exacerbated inflammatory responses after SARS-Cov-2 infection. These results along with transcriptome analysis of DNA repair genes will define lower NEIL2 expression as a predictive biomarker, and thereby stratifying risk groups.

### Example 2

### The Specific Binding of NEIL2 to 5′-UTR of CoV-2 Genomic RNA Inhibits CoV-2 Replication

SARS-CoV-2 contains positive-sense, single-stranded RNA genome which is directly translated by hijacked host protein synthesis machinery to synthesize non-structural proteins (NSPs). NSPs play a critical role in shutting down host protein synthesis and optimizing cellular condition for viral mRNA synthesis and virus replication. The viral pathogenicity depends on the ability to actively reprogram the host cell metabolism or suppress host antiviral defense mechanisms. Conversely, host cells activate their defense machinery to restrict viral infection. The preliminary studies implicating NEIL2 as a host factor with the critical role of suppressing viral infection via its specific binding to 5′-UTR of SARS-CoV-2 will be expanded upon.

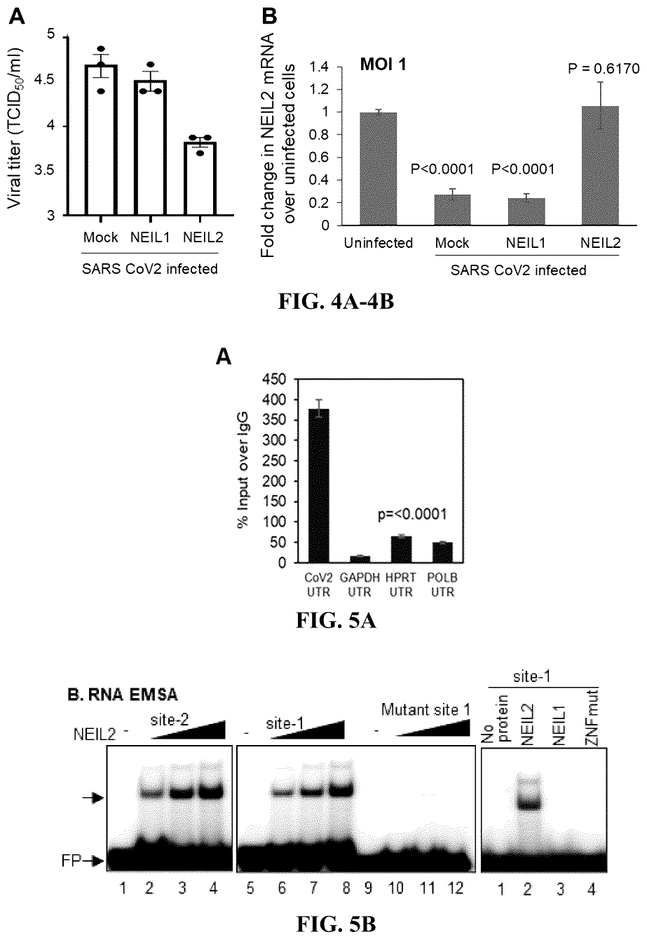

Preliminary Studies. (a) Transduced rNEIL2 inhibits CoV-2 replication. In view of emerging success of protein replacement therapy^{36,37}, transduced rNEIL2 was tested for providing protection from SARS-CoV-2 infection in human A549-ACE2 alveolar epithelial cells. A manufacturers protocol (Pierce™) was used to show that rNEIL2 (1 μg/10⁶ cells), but not rNEIL1 transduced in A549-ACE2 cells, 24 hrs prior to infection with SARS-CoV-2 (1 MOI) significantly inhibited SARS-CoV-2 viral replication (FIG. 4A). Notably, the endogenous level of NEIL2's transcript was significantly decreased in mock and rNEIL1, but not rNEIL2 transduced A549-ACE2 cells (FIG. 4B), supporting NEIL2's unique function in inhibiting SARS-CoV-2 replication.

(b) NEIL2 binds at the 5′-UTR of CoV-2 and regulates viral protein synthesis. Activation of the antiviral innate immune signaling cascade generally begins with the recognition of viral genomes by intracellular pattern recognition receptors^{38,39} or by a set of zinc finger proteins (ZFPs), such as zinc-finger antiviral proteins (ZAP) that can lead to viral suppression through translation or replication repression⁴⁰. Given that NEIL2 contains a CHCC type zinc finger motif⁴¹ that enables it to bind both double and single stranded DNA and that it showed antiviral properties against SARS-CoV-2 infection (FIG. 4), NEIL2 interaction with SARS-CoV-2 RNA was tested. A SARS-CoV-2 5′-UTR containing GFP expression plasmid was transfected in BEAS-2B (human lung epithelial) cells and then performed RNA ChIP using anti-NEIL2 antibody following a published protocol^{42,43} Indeed, strong association of NEIL2 with full length SARS-CoV-2 5′-UTR was detected, but not with the 5′-UTR of several host genes (GAPDH, HPRT and DNA polymerase (3), as controls, in the RNA ChIP analysis (FIG. 5A). Using RNA binding motif search tools, two zinc finger (ZnF) binding sites (site-1, nt 148-172 and site-2, nt 209-233) were identified in the SARS-CoV-2 5′-UTR. EMSA showed NEIL2's robust sequence specific and dose-dependent binding to both of these sites (FIG. 5B, site-1, lanes 6-8; site-2, lanes 2-4), but not to the control or mutant RNA (lns 10-12). Inability of the binding of NEIL1 or of the ZnF mutant NEIL2 to the 5′-UTR sites of CoV-2 underscored the specificity of NEIL2's RNA binding (FIG. 5B, Right panel). To assess whether such binding affects SARS-CoV-2 replication, RNA dependent RNA polymerase (RdRp, nsp12 complex with accessory nsp7-nsp8) activity was tested using CoV-2 5′-UTR-ZnF motif containing RNA oligo as the template (site-1 and -2) and short complementary oligo sequences as primers in the presence or absence of rNEIL2 to initiate 5′-3′ extension, following the protocol as described 44. However, a significant effect of NEIL2 on viral RdRp activity in vitro could not be detected. The inventors contemplate that NEIL2 regulates viral protein synthesis by blocking activity of host translational machinery at the 5′-UTR of SARS-CoV-2.

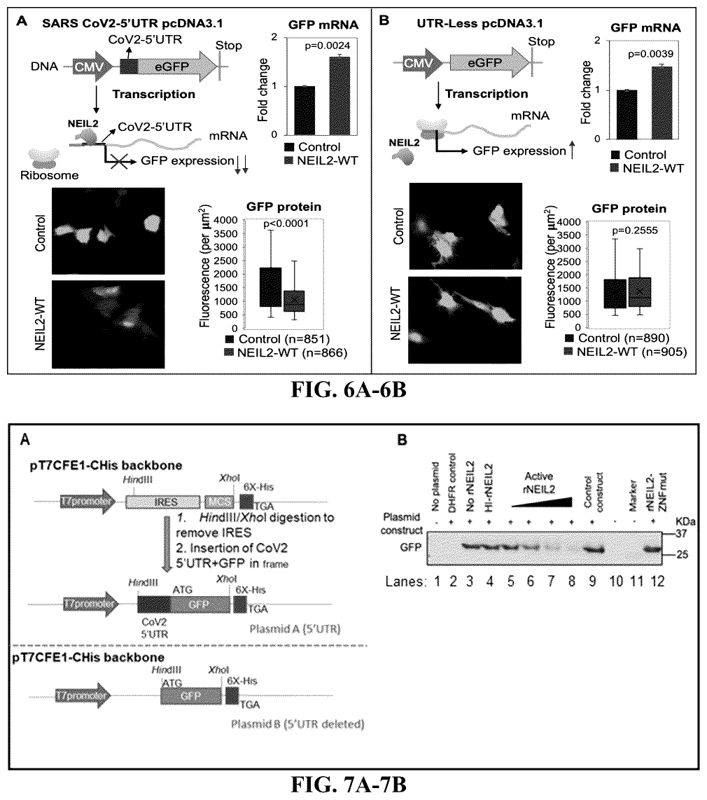

To verify the regulatory function of NEIL2, GFP expression plasmids under the control of a CMV promoter were constructed with or without the 5′-UTR (˜300nt long) of SARS-CoV-2 as shown in FIG. 6A. NEIL2 overexpressing vs control human lung epithelial BEAS-2B cells (n=3, biological replicate) were transfected with the engineered construct and GFP expression was monitored. GFP expression was found to be significantly reduced at the protein level (FIG. 6A, lower panel), but not at the transcript level (upper panel), in NEIL2 overexpressing cells compared to control. Notably, deletion of 5′-UTR did not show this reduction (FIG. 6B).

To further confirm NEIL2's inhibitory role, a reconstituted in vitro mammalian expression system containing all purified components needed for coupled transcription and translation (Pierce) was used. The effect of purified rNEIL2 was tested, as per the manufacturer's protocol, on GFP expression from a plasmid in which the original internal ribosome entry site (IRES) was replaced with 5′-UTR of SARS-CoV-2 (FIG. 7A). FIG. 7B clearly shows NEIL2's dose-dependent (Lanes 3, 4 vs 5-8; 25-100 ng) inhibition of GFP protein expression. However, heat inactivated (HI) or ZnF mutant NEIL2 (Lanes 4 and 12, 100 ng) had no effect. Collectively, these data strongly support the scenario that cytosolic NEIL2 provides an important protective role by blocking utilization of the host protein synthesis machinery, for viral replication.

Based on the above preliminary data, an experimental plan was developed as detailed below.

Experimental design. (a) To test the hypothesis that NEIL2 interferes with the synthesis of CoV-2 proteins:

(i) Identify the NEIL2 RNA binding domain (RBD) and analyze protein-RNA (5′-UTR) interactions. Although NEIL1 and NEIL2 are related DNA oxidized base repair proteins, the preliminary data showed that NEIL2, but not NEIL1, specifically binds to SARS-CoV-2 viral RNA at the 5′-UTR. Recent structural studies have identified the DNA binding motif of NEIL245; however, its RNA binding domain (RBD) has not been characterized. The inventors have cloned and expressed naturally occurring isoforms of NEIL2 (per NCBI data base) and will further characterize the domain/motif of NEIL2 that specifically binds to the SARS-CoV-2 5′-UTR. The binding affinity of NEIL2 for the SARS-CoV-2 5′-UTR will also be determined, which would help evaluate the strength of bimolecular interaction.

(ii) In vitro transcription-coupled translation assay. FIG. 7 clearly shows that WT rNEIL2 inhibited protein synthesis from viral RNA. The effect of purified DNA repair deficient (K50R), ZnF, and RBD domain mutants of rNEIL2 in vitro will be tested in reconstituted transcription-coupled translation assay. These studies will identify the specific activity of NEIL2 that is necessary for blocking viral protein synthesis, and consequently its replication.

(iii) RNA ChIP-qPCR analysis. RNA-ChIP analysis is a highly valuable technique for analyzing RNA-protein interaction. RNA ChIP analysis will be performed with anti-NEIL2 antibodies to examine its association with the SARS-CoV-2 5′-UTR in viral infected cells. Once confirmed which of the isoforms/mutants binds to the 5′-UTR by in vitro gel shift analysis (as proposed), RNA ChIP will be performed from the cells stably expressing the particular mutant (K50R, ZnF or RBD mutant). Variant cells have been generated with a FLAG-tag to be used for RNA-ChIP analysis. Additionally, The affinity of NEIL2 binding to the specific sequence within 5′-UTR will be measured. Collectively, these studies will help understand the molecular mechanism of NEIL2-mediated inhibition of viral replication.

(b) Analysis of NEIL2 binding to CoV2-5′-UTR. To analyze binding of NEIL2 to 5′-UTR, biotinylated SARS-CoV2-5′-UTR (300 nt) will be transcribed in vitro to test its binding of WT vs. inactive variants of NEIL2. Moreover, Biotin labelled RNA pulldown from NEIL2 expressing vs. NEIL2 depleted cells will be subjected to mass spectrometry (MS) analysis to test if NEIL2 (along with other proteins) are bound to the SARS-CoV-2 5′-UTR. Several publications reported on the SARS-CoV-2 RNA-protein interactome⁴⁶⁻⁴⁸; however, NEIL2 was not identified as one of the candidate proteins. One possible reason could be depletion of NEIL2 due to its SARS-CoV-2 infection-induced downregulation. Because viral infection downregulates NEIL2, only biotinylated SARS-CoV2-5′-UTR will be used and pull down the complex from WT vs NEIL2-depleted cells using streptavidin beads for subsequent MS identification of the bound proteins. This study would provide valuable insight about how NEIL2 affects host protein binding to the 5′-UTR. Once the NEIL2 RNA binding domain (RBD) is identified, the mutant protein lacking the RBD will be transfected in A549-ACE2 cells and examine if the mutant protein fails to protect against viral infection as shown previously (FIG. 4).

(c) How NEIL2 is targeted for degradation? NSPs of SARS-CoV-2 are multifunctional. In addition to their role in suppression of the host mRNA translation, they also target host mRNA for degradation in which NSP1 was specifically proposed to play a critical role 49. NSP1-FLAG will be ectopically expressed and preform RNA ChIP by using anti-FLAG Ab and analyze if the pre-mRNA of NEIL2 is preferentially bound to NSP1. NEIL2's transcript level will be monitored in NSP1 transfected cells. This experiment will provide information about NSP1's role in NEIL2 mRNA stability.

Expected Results. The inventors expect that WT NEIL2, its active site mutant (K50R) or a variant, but not the RBD mutant, will block viral protein synthesis. These studies will establish that the non-repair function of cytoplasmic NEIL2 plays an important role in mitigating viral replication.

### Example 3

### Administration of rNEIL2 Will Attenuate SARS-CoV-2 Infection and Disease in Well-Characterized Animal Models

Rationale. It has been demonstrated that (1) Transduction of rNEIL2 in human A549-ACE2 cells attenuates SARS-CoV-2 infection (FIG. 4) as elaborated later in the Preliminary Studies. (2) Intranasal administration of rNEIL2 greatly prevented the weight loss and viral load in RSV-infected Neil2^{-/-} mice. Thus, further studies will focus on confirming rNEIL2 as an antiviral agent against SARS-CoV-2 infection and disease in permissive mouse and hamster models. These are well-characterized and routinely used in the inventors' laboratory for the development of effective medical countermeasures (MCMs) against COVID-19. Multiple endpoints directly associated with CoV-2 infection, including viral loads and histopathology of tissues, morbidity, weight changes and other signs of illness, mortality, and tissue inflammatory responses, will be pursued for assessing the protective efficacy of rNEIL2. Among several models that are readily available in the inventors' lab, initially Neil2^{-/-} or BALB/c mice/mouse-adapted CoV-2-MA10 will be used, followed by hACE2 Tg mice/SARS-CoV-2(US WA-1/2020) clinical isolate. Additionally, BALB/c mice will be used for testing the efficacy of rNEIL2 against selected VOC strains, such as β- and γ-variants. Finally, hamsters are used as an additional model to evaluate the protective potential of rNEIL2 against WA-1/2020 and other VOC strains, including α-, β-, γ-, δ-, δ-plus, μ-, and, recently, Omicron isolates, if needed.

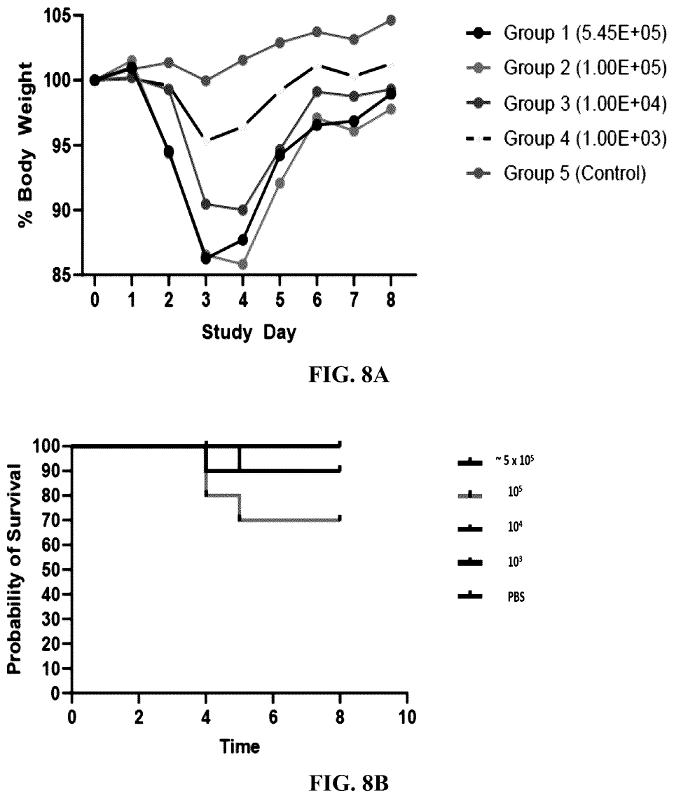

Preliminary studies: (a) Neil2^{-/-} mice were significantly more susceptible to SARS-CoV-2-MA10 infection than Neil2^{+/+} mice. To investigate the biological significance of NEIL2 in COVID-19 pathogenesis, a Neil2-null mouse model developed in the inventors' lab was used¹⁷. While clinical isolates of SARS-CoV-2 WA1/2020 could not readily infect wild-type (WT) BALB/c or C57BL/6 mice, mouse-adapted (MA) SARS-CoV-2-MA10 could productively infect WT BALB/c mice (FIG. 8) and C57BL/c mice (data not shown), resulting in weight loss and mortality in an age-dependent manner, as originally reported (Tseng et al., unpublished and⁵³). Therefore, this mouse adapted viral strain provided the opportunity to explore the impact of viral infection in WT vs Neil2-null mice as summarized below.

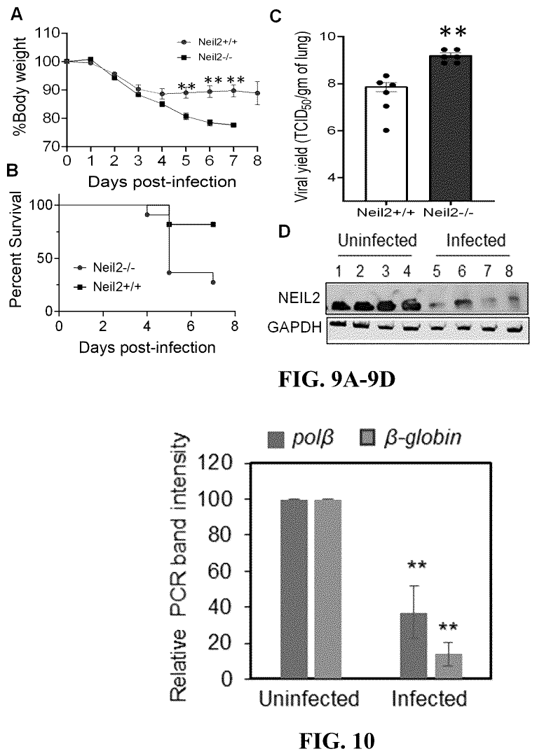

Neil2^{+/+} and Neil2^{-/-} mice (˜6 to 7-months old) were challenged intranasally (i.n.) with 2×10⁵ CoV-2-MA10 particles and monitored daily for the onset of morbidity (i.e., weight changes and other signs of illness) and mortality (if any). It was noted that infected Neil2^{-/-} mice were losing weight more aggressively than the WT cohort (FIG. 9A) concomitant with the onset of other signs of illness (data not shown). More importantly, 80% of Neil2^{-/-} mice succumbed to infection within 4-6 days compared to 20% mortality in Neil2^{+/+} mice (FIG. 9B). Yields of infectious virus within the lung of Neil2^{-/-} mice were significantly higher (˜20-folds) than that of Neil2^{+/+} mice at 2 dpi (FIG. 9C). Consistent with earlier studies CoV-2-MA10 infection caused significant downregulation of NEIL2 (FIG. 9D). This study clearly indicates that NEIL2 plays a protective role against CoV-2 infection.

(b) DNA damage analysis in CoV-2 infected mice lungs. To elucidate the effect of SARS-CoV-2-infection induced hyper inflammation on the host genome, DNA damage by LA-qPCR in uninfected vs CoV2-MA10 infected Neil2^{+/+} mice were analyzed (FIG. 10). Briefly, genomic DNA was extracted from the lung tissues of Neil2^{+/+} mice 5 days after infection, and gene-specific long amplicon qPCR (LAqPCR) was performed, a routine procedure in the inventors' lab^{17,42,43}. It was found that CoV2-MA10 infected Neil2^{+/+} mice do accumulate ˜3-5 fold more DNA damage (FIG. 10). Of note, DNA damage in Neil2^{-/-} mice could not be measured because those (80%) died within 5 days. These data thus suggest that virus infection induced genome damage plays a role in COVID-19 pathogenesis.

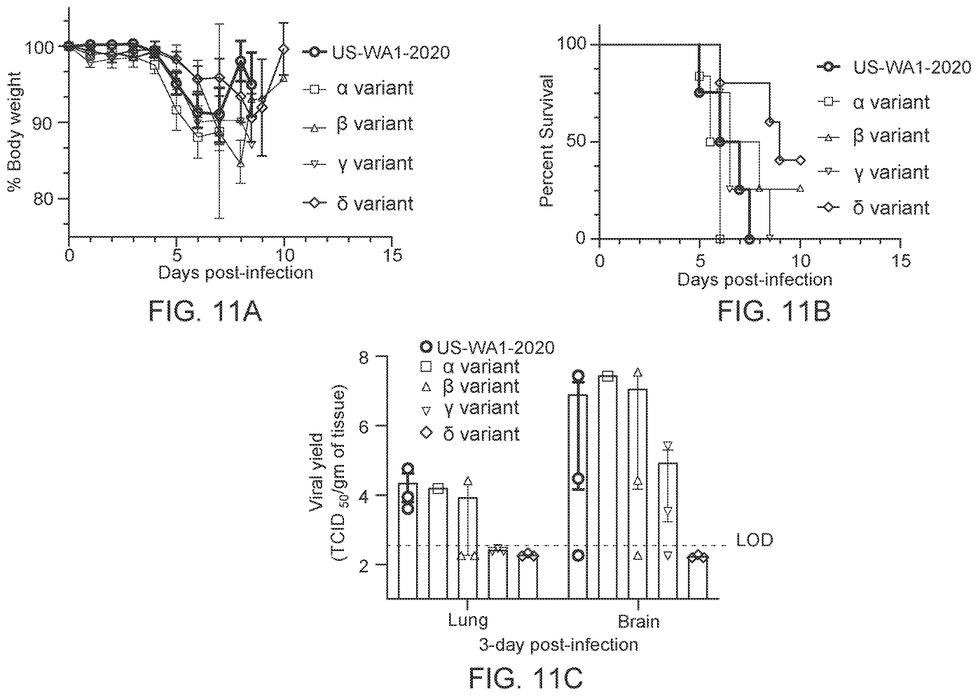

(c) Human ACE2 Tg and BALB/c mice are susceptible to clinical isolates of SARS-CoV-2 and VOC strains. The inventors have established and then extensively characterized Tg mice expressing hACE2 or hDPR4 that are fully permissive to SARS-CoV and MERS-CoV, respectively, resulting in acute onset of morbidity (i.e., weight loss and other signs of illness) and mortality⁵⁴⁻⁵⁷. Such a uniform morbidity and mortality of both Tg mouse strains in response to SARS-CoV and MERS-CoV infection offers convenient and unbiased endpoints, making them popular and successful preclinical animal models for assessing the efficacy and safety of medical countermeasures (MCMs). Like SARS-CoV, the newly emerged SARS-CoV-2 uses the same hACE2 as an entry receptor to initiate infection in permissive hosts. To promptly meet the urgent need for an animal model to study SARS-CoV-2 pathogenesis and development of MCMs, under the NIH/NIAID's support (i.e., A38 Task Order), the inventors have succeeded in comprehensively characterizing one of the hACE2-Tg mouse lineages, AC70, as a robust model for COVID-19, with values of LD₅₀ and ID₅₀ of merely ˜3.2 and ˜0.5 TCID₅₀ of SARS-CoV-2 (US-WA1/2020), respectively (data not shown). As expected, AC70 ACE2-Tg mice were also permissive to UK (α), SA (β), Brazil (γ), and Indian (δ) strains of Variants of Concern (VOC), causing weight loss and mortality with varying kinetics and intensities (FIG. 11). This well-characterized AC70 ACE2-Tg mouse model has been routinely and successfully used in the inventors' lab for assessing both preventive and therapeutic MCMs against COVID-19 (data not shown).

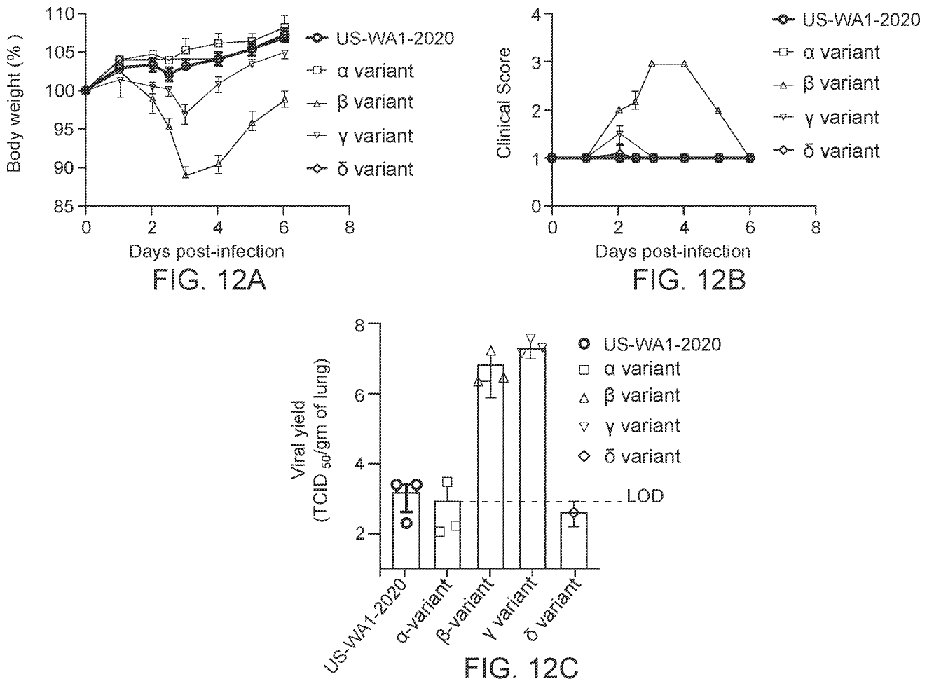

Additionally, the inventors have shown that some of the VOC strains, especially β- and γ-variants, could directly infect BALB/c mice without prior adaptation in vivo, resulting in the onset of weight loss and other signs of illness (FIG. 12). Briefly, groups of 8 BALB/c mice were challenged (i.n.) with indicated doses of WA-1, α-, β-, γ-, and δ-variants, respectively. Three mice in each group were euthanized at 2 dpi for determining viral loads and histopathology of the lungs, the remaining 5 animals were subjected to daily monitoring for the onset of morbidity and mortality (if any). While the histopathology of infected mice is under investigation, the onset of clinical scores (FIG. 12A) and weight loss (FIG. 12B) were noted in mice infected with either β- or γ-variants, along with high yields of live virus recovered at 2 dpi (FIG. 12C). Thus, in addition to AC70 hACE2 Tg mice, BALB/c mice can also be used to assess the drug efficacy directly against selected VOC strains without the need of any prior adaptation.

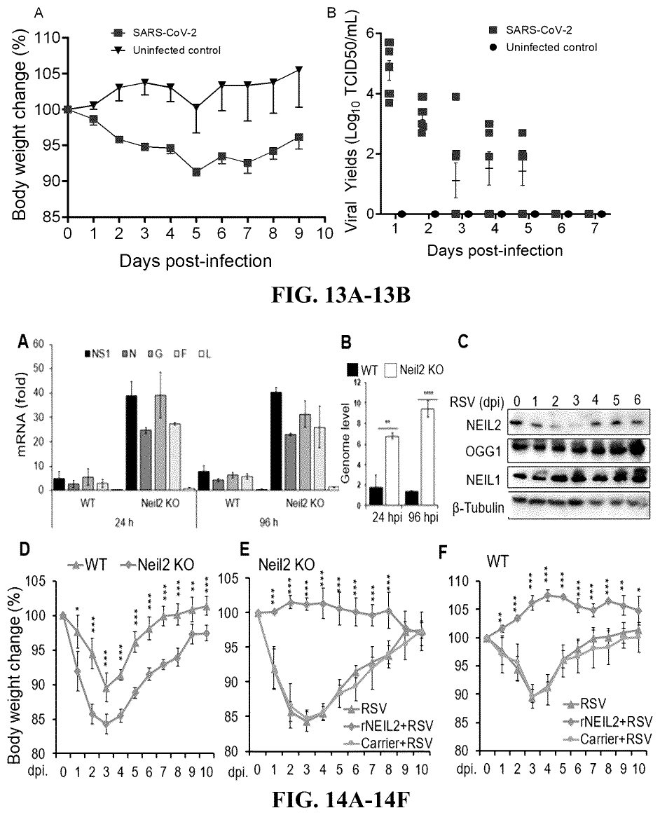

(d) Hamsters as an alternative animal model for COVID-19 pathogenesis. The inventors confirmed published reports that golden Syrian hamsters are permissive to SARS-CoV-2 infection (FIG. 13)⁵⁸⁻⁶⁰. This was reflected in intense virus replication and persistent weight loss after infection. Briefly, two groups of 10 female hamsters (7-8-weeks old, Charlies River) were challenged (i.n.) with 1×10⁶ TCID₅₀ of SARS-CoV-2 (USA WA-1/2020 isolate). Animals were monitored for morbidity and mortality up to 9-dpi. SARS-Cov-2 infected hamsters started losing weight from 1-dpi and maximum weight loss of ˜10% at 5-dpi (FIG. 13A). Nasal swabs of hamsters from 1-7-dpi collected in PBS were analyzed for live virus using VeroE6 cells with standard viral titration assay. High level of live virus was detected up to 5 dpi with peak viral shedding at 1 and 2-dpi (FIG. 13B).

(e) Administration of rNEIL2 significantly ameliorated body weight loss and RSV replication in Neil2^{-/-} mice. The respiratory pathogen, RSV, causes symptoms similar to SARS-CoV-2 in children, elderly and in high-risk individuals with comorbidity. Recent studies have shown that 3-4% of severe COVID-19 patients also have co-infection with RSV 61. RSV was used as a model and tested the effect of NEIL2 on RSV gene expression and subsequent weight loss (FIG. 14). Briefly, Neil2^{+/+} and Neil2^{-/-} mice were infected (i.n.) with 1×10⁶ PFU of purified RSV and the lungs were harvested at 24 h and 96 h after infection. FIG. 14A shows that the transcript levels of the viral genes (NS1, N, G, F and L) were increased more than 20-fold in Neil2^{-/-} at both 24 h and 96 h post RSV infection compared to WT (Neil2^{+/+}). Significant increase was also observed in the viral genomic levels in Neil2^{-/-} lungs (FIG. 14B). These results are consistent with those of earlier studies showing RSV infection caused significant downregulation of NEIL2 in WT mice (FIG. 14C). Moreover, weight loss in Neil2-null mice was significantly higher than in WT mice (FIG. 14D). The inventors recently reported that intranasal delivery of rNEIL2 into mouse lung mitigated TNFα-induced inflammatory response¹³. Using this method of protein delivery, the inventors surprisingly observed that a single dose of rNEIL2 (15 μg) provided strong protection against RSV infection in both Neil2-null (FIG. 14E) and WT mice (FIG. 14F). NEIL2 thus is likely to play a broader role in protecting the experimental animals and human population from various types of viral infection.

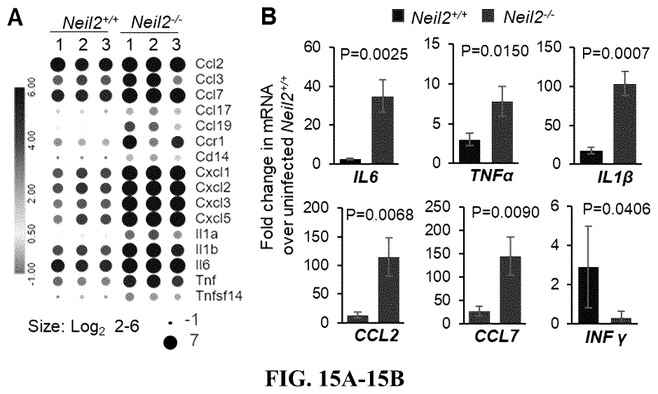

(f) Neil2^{-/-} mice are more susceptible to CoV-2-MA10-induced inflammatory mediators. Given that NEIL2 is significantly downregulated in SARS-CoV-2 infected patients, as well as in both hamster and mice, a multiplex RT-qPCR analysis of 84 inflammation-associated genes in lungs of CoV2-MA10 infected Neil2^{-/-} vs. Neil2^{+/+} mice was conducted. Infected Neil2^{-/-} lungs had elevated expression of ˜47 genes and decreased expression of ˜8 genes more than 2-fold compared to infected Neil2^{+/+} animals. The inventors observed increased expression of genes involved in natural killer (NK) cells or monocyte trafficking (CCL2 and CCL3), neutrophil trafficking (CXCL1 and CXCL2), as and in the regulation of immune response, hematopoiesis, or inflammation (TNFα, IL6 and IL1β) causing inflammation of lungs, which is strikingly similar to the cytokine storm observed in mild to severe COVID-19 patients^{8,62,63}. However, a significant decrease was observed in anti-viral cytokine IFNγ (p=0.0406). FIG. 15A shows a subset of proinflammatory genes significantly increased in infected Neil2^{-/-} mouse lung (n=3) compared to mock treated Neil2^{+/+} mouse lung. The multiplex assay results were further validated for the subset of significantly altered genes by RT-qPCR analysis of RNA from the mouse lungs (FIG. 15B). Collectively, these data indicate that NEIL2 deficiency could lead to exacerbated CoV-2 infection (FIG. 9), resulting in uncontrolled inflammatory response (FIG. 15).

Experimental designs. As detailed below, the proposed in vivo studies are designed to achieve reproducible and rigorous results. The models chosen were selected because they have been well established and widely used to study pathogenesis and candidate treatments for human coronavirus infections. All animal studies will be conducted in accordance with the ARRIVE (Animal Research: Reporting In Vivo Experiments) guidelines strictly based on approved IACUC and ACURO protocols.

(a) To test the hypothesis that intranasal delivery of WT rNEIL2 will attenuate SARS-CoV-2 infection. Table 1 describes an overall study design for testing prophylactic and therapeutic efficacy of rNEIL2 protein against SARS-CoV-2 infection and disease in vivo. The inventors have demonstrated in preliminary studies that intranasal administration of a single 15 dose of rNEIL2/mouse at 48 h before respiratory challenge with RSV effectively prevented viral infection and weight loss (FIG. 14). The same dosing strategy will be adopted, i.e., 15 μg of rNEIL2 via i.n. route in mice, to test whether such a single-dose treatment either before or after SARS-CoV-2 infection could attenuate SARS-CoV-2 infection and disease in permissive hosts. Based on ˜4-5-times higher weight of hamsters than mice, each hamster will initially be treated with ˜75 μg rNEIL2/i.n and further optimize the dose if necessary. The DNA repair deficient and RBD mutant rNEIL2 will be used as a control in this study. Specifically, rNEIL2 in Neil2^{+/+}, Neil2^{-/-}, and BALB/c mice/MA10 models and hACE2 Tg mice and hamsters/clinical isolates of SARS-CoV-2 models will be used to explore the usage of rNEIL2 as a novel antiviral agent against CoV-2.

TABLE 1
Prototypic study design for testing the protective
efficacy of rNEIL2 against SARS-CoV-2 infection

| | No | | | | |
|---|---|---|---|---|---|
| | animals/ | | Treatment | Treatment | |
| Groups | group | Compounds | time IN* | volume (μl) | Challenge dose/vol/route |
| 1 | 10 | Vehicle | 48 h pre- | 60 (mouse) or | 10⁵ TCID₅₀ of MA10, |
| | | | challenge | 100 (hamster) | WA-1**, 2 selected |
| 2 | | WT or mutant rNEIL2 | 48 h pre- | | VOC variants for BALB/c, |
| | | (15 μg/mouse or | challenge | | NEIL2^{+/+ or -/-} mice |
| 3 | | 75 μg/hamster) | 4 h post- | | and hamsters Or |
| | | | challenge | | 5 TCID₅₀ (~1.7 LD50) of |
| 4 | | | 12 h post- | | WA-1 or 2 selected VOC |
| | | | challenge | | variants for hACE2 Tg |
| 5 | | | 24 h post- | | in 60 or 100 μl/IN |
| | | | challenge | | for mice and hamsters |

*IN: Intranasal route;
**WA-1: SARS-CoV-2(US_WA-1/2020) isolate

In addition to the original isolate of SARS-CoV-2(US WA-1/2020), the inventors have a rich collection of VOC mutants, including α-, γ-, δ-, δ⁺-, μ-, and, recently, Omicron variants, to further testing the protective efficacy of rNEIL2 against Pan-CoV-2 viruses. Briefly, groups of 10 animals will be treated once with rNEIL2 or vehicle at either 48 h before or 4 h, 12 h, or 24 h post challenge with indicated doses of MA10 or WA-1 and up to two “to-be-selected” VOC strains (e.g., -β and γ-strains). Infected animals will be monitored at least daily for the onset of morbidity (weight loss and other signs of illness) and mortality for up to 14 dpi. Four mice in each group will be euthanized at a selected time point, putatively between 2-4 dpi, depending on the status of clinical disease, for harvesting lung, brain, and other tissues for assessing histopathology and determining viral loads by the standard Vero E6-based infectivity and qPCR-based assays. Together, the parameters including viral loads, histopathology, weight loss, and clinical scores will be used as the endpoints for assessing the efficacy of rNEIL2 against CoV-2. Based on the inventors extensive experience, hamsters of 6-8-weeks old are naturally permissive to SARS-CoV-2 infection with prominent viral infection within the upper respiratory tract before descending to the lower respiratory tract (i.e., lungs), concomitant with transient but increasing weight loss that usually occurs between 1-5 dpi before gradually regaining weight. Thus, the weight changes and other symptoms of illness (if any) will be monitored daily, collect nasal washes of infected hamsters at least at 1, 2, and 3 dpi for assessing viral loads (both live virus and viral RNA). Five hamsters of each group will be euthanized at 3 and 7 dpi, respectively, for assessing viral loads and histopathology of the lungs. Once the optimal time of treatment is selected, the study will be repeated at least once with two groups of 10 animals/experimental group to confirm the protective efficacy of rNEIL2 against Pan-CoV-2 infection and disease.

(b) DNA damage, and transcriptome analysis of DNA repair and inflammatory genes in CoV2-MA10 infected animal lungs. As viral infection-induced accumulation of DNA damage can modulate transcription, genome damage will be first analyzed (both nuclear and mitochondrial) by LA-qPCR in lung tissues as well as PBMC from CoV2-MA10 infected Neil2^{-/-} vs Neil2^{+/+} mice and uninfected controls post ±rNEIL2 transduction. The inventors will then perform transcriptomic analysis of the DNA repair genes in the isolated RNA from same tissues by RNA-seq. The inventors will continue to validate the level of NEIL2 and as controls (such as OGG1, NEIL1, NEIL3, NTH1 and MYH1,) by gene-specific qRT-PCR. These data will be correlated with body weight loss and immune responses of the animals and corroborate these findings with the data derived from the analysis of PBMC.

Mounting evidence clearly indicate that uncontrolled inflammation in combination with the lack of sufficient interferon (IFN) responses against SARS-CoV-2 at the early stage of infection, causes patients to succumb rapidly to COVID-19. IFN are protective; however, if homeostasis is not maintained then sustained activation would cause tissue damage leading to organ failure and could be fatal. The major candidate inflammatory cytokines have already been well characterized^{64,65}. To further corroborate those studies transcriptome data will be analyzed. In brief, total RNA will be isolated from lung tissues (Neil2^{-/-} vs. Neil2^{+/+} mice ±rNEIL2) and inflammatory gene expression will be determined using RT2 Profiler PCR Array (Mouse Inflammatory Cytokines & Receptors). The inventors will further validate the expression of critical genes by RT-PCR. Additionally, ELISA (Bio-Plex31, BioRad) will be performed to quantify inflammatory chemokines/cytokines at the protein level. These results will be helpful in identifying differences among Neil2^{-/-} vs. Neil2^{+/+} mice ±rNEIL2 in the proinflammatory mediator (s), inhibition of which along with delivery of rNEIL2 would be more beneficial.

Statistical Plan/analysis. An unpaired, two-tailed Student's t-test (for parametric data) or Mann-Whitney test (for nonparametric data) will be used to analyze the significance of two data sets (GraphPad Prism); P≤0.05 or ≤0.01 will be considered significant or highly significant, respectively. One-way ANOVA (with Tukey HSD post hoc test) will be used to compare more than two groups or a Kruskal-Wallis with post hoc Dunn's test (for nonparametric data) for multiple comparisons. Kaplan-Meier and Log-Rank (Mantel-Cox) pairwise comparison test will be used for analysis of mouse survival. Based on our prior experience, when using morbidity and mortality as the endpoint for determining the efficacy of MCMs, a sample size of 10 in each group has a 90% power to detect an increase in survival proportion of 0.569 with a significance level of 0.05 (two-tailed). If infectious viral titers and histopathology of the lungs are needed as additional endpoints, we will use six animals in each group and/or time point for comparisons. A sample size of 6 animals in each group and/or time point will allow for the detection of a difference in geometric viral titer of 4-fold (0.6 log10) or greater between groups, assuming a standard deviation of 0.3 logs, using a two-sample t-test and a two-tailed alpha of 0.05.

Sex and biological variables. Although the inventors are yet to observe any significant difference between male and female hACE2 and BALB/c mice in response to SARS-CoV-2 infection, we will use an equal number of each sex for all experiments. We have collected at least 10 different clinical isolates of SARS-CoV-2, including all major strains of Variants of Concern (VOC) over the past few months, including δ⁺, μ-, and Omicron. This rich collection will enable us to test the efficacy of rNEIL2 against heterologous strains of pandemic CoV-2.

Reproducibility and Rigor. Data reproducibility and rigor will be ensured by performing independent repeats for each proposed experiment, and biological replicates will be included in the experimental design for achieving statistical significance. Experimental quality and validity will be ensured by the inclusion of positive and negative controls for all assays. Data integrity will be ensured by the PIs via review of notebook entries.

Biosafety: All animal studies proposed in this application employing SARS-CoV-2 will be done under animal BSL-3 conditions at the Galveston National Laboratory BSL3 and ABSL3 facility at UTMB.

### REFERENCES

1. Planas, D. et al. Nature, doi:10.1038/s41586-021-04389-z (2021).
2. Hu, J. et al. doi:10.1038/s41423-021-00836-z (2022).
3. Sinha, P. et al. Int J Infect Dis 99, 28-33, doi:10.1016/j.ijid.2020.07.023 (2020).
4. Xu, X. et al. PNAS USA 117, 10970-10975, doi:10.1073/pnas.2005615117 (2020).
5. Veiga, V. C. et al. BMJ 372, n84, doi:10.1136/bmj.n84 (2021).
6. Rosas, I. O. et al. N Engl J Med 384, 1503-1516, doi:10.1056/NEJMoa2028700 (2021).
7. Hariharan et al., Inflammopharmacology 29, 91-100, doi:10.1007/s10787-020-00773-9 (2021).
8. Zhong et al., Lancet Rheumatol 2, e428-e436, doi:10.1016/S2665-9913(20)30120-X (2020).
9. Ye et al., Respir Med Case Rep 30, 101087, doi:10.1016/j.rmcr.2020.101087 (2020).
10. Bordag et al. Scientific reports 5, 15954, doi:10.1038/srep15954 (2015).
11. Tomazini et al. JAMA 324, 1307-1316, doi:10.1001/jama.2020.17021 (2020).
12. Group et al., N Engl J Med 384, 693-704, doi:10.1056/NEJMoa2021436 (2021).
13. Tapryal et al., The Journal of biological chemistry 296, 100723, (2021).
14. Hazra et al. PNAS USA 99, 3523-3528. (2002).
15. Hazra et al. The Journal of biological chemistry 277, 30417-30420. (2002).
16. Banerjee et al., The Journal of biological chemistry 286, 6006-6016 (2011).
17. Chakraborty et al., The Journal of biological chemistry 290, 24636-24648 (2015).
18. Vlahopoulos et al., Cells 10, doi:10.3390/cells10113067 (2021).
19. Pan et al., Scientific reports 7, 43297, doi:10.1038/srep43297 (2017).
20. Visnes et al., Science 362, 834-839, (2018).
21. Huang et al., Lancet 395, 497-506, (2020).
22. Chen et al., The Journal of clinical investigation 130, 2620-2629, (2020).
23. Hirano and Murakami, Immunity 52, 731-733, doi:10.1016/j.immuni.2020.04.003 (2020).
24. Sestakova et al. Mutat Res Genet Toxicol Environ Mutagen 854-855, 503200, (2020).
25. Pariset et al., Cell reports 33, 108434, (2020).
26. Perico et al., Nat Rev Nephrol 17, 46-64, (2021).
27. Nasi et al., Toxicol Rep 7, 768-771, (2020).
28. Jiang and Mei, Viruses 13, (2021).
29. Saleh et al., Mitochondrion 54, 1-7, (2020).
30. McDonald et al., Cell reports 37, 109839, (2021).
31. Alfarouk et al., J Enzyme Inhib Med Chem 36, 1258-1267, (2021).
32. Mandal et al., The Journal of biological chemistry 287, 2819-2829 (2012).
33. Ye et al., Scientific reports 10, 5136, (2020).
34. Dey et al., DNA repair 11, 570-578 (2012).
35. Barra et al., Diagnostics (Basel) 9, (2019).
36. Gorzelany and de Souza, Sci Transl Med 5, 178fs110, (2013).
37. Tambuyzer et al., Nat Rev Drug Discov 19, 93-111, (2020).
38. Ablasser and Hur, Nat Immunol 21, 17-29, (2020).
39. Rehwinkel and Gack, Nat Rev Immunol 20, 537-551, (2020).
40. Lin et al., Nucleic acids research 48, 7371-7384, (2020).
41. Das et al., The Journal of biological chemistry 279, 47132-47138, (2004).
42. Chakraborty et al., Nature communications 7, 13049, (2016).
43. Chakraborty et al., PNAS USA 117, 8154-8165, (2020).
44. Gordon et al., The Journal of biological chemistry 295, 6785-6797, (2020).
45. Eckenroth et al., Structure 29, 29-42 e24, (2021).
46. Schmidt et al., Nat Microbiol 6, 339-353, (2021).
47. Kamel et al., Molecular cell 81, 2851-2867 e2857, (2021).
48. Flynn et al., Cell 184, 2394-2411 e2316, (2021).
49. Thoms et al., Science 369, 1249-1255, (2020).
50. Miao et al., RNA biology 18, 447-456, (2021).
51. Squeglia et al., Front Chem 8, 602162, (2020).
52. Ciccosanti et al., Antiviral Res 190, 105064, (2021).
53. Sheahan et al., Sci Transl Med 12, doi:10.1126/scitranslmed.abb5883 (2020).
54. Agrawal et al., Journal of virology 89, 3659-3670, (2015).
55. Tao et al., Journal of virology 90, 57-67, (2016).
56. Tseng et al., Journal of virology 81, 1162-1173, (2007).
57. Yoshikawa et al., Journal of virology 83, 5451-5465, (2009).
58. Sia et al., Nature 583, 834-838, (2020).
59. Chan et al., Clin Infect Dis 71, 2428-2446, (2020).
60. Imai et al., PNAS USA 117, 16587-16595, (2020).
61. Lansbury et al., J Infect 81, 266-275, (2020).
62. Liao et al., Nat Med 26, 842-844, (2020).
63. Gong et al., BMC Infect Dis 20, 963, (2020).
64. Ragab et al., Front Immunol 11, 1446, (2020).
65. Tang et al., Front Immunol 11, 570993, (2020).
66. Bojkova et al. Nature 583, 469-472, (2020).
67. Bastard et al., Science 370, (2020).

1. A method for treating a virus induced dysregulated inflammatory response in a subject in need of such treatment, the method comprising the step of administering to the subject a therapeutically effective amount of a NEIL2 peptide or a composition comprising the NEIL2 peptide, wherein the treatment inhibits and/or ameliorates at least one symptom of the virus induced dysregulated inflammatory response.

2. The method of claim 1, wherein the NEIL2 peptide or the composition comprising the NEIL2 peptide is administered by inhalation or instillation to the lungs.

3. The method of claim 1, wherein the NEIL2 peptide or the composition comprising the NEIL2 peptide is administered intranasally.

4. The method of claim 1, wherein the NEIL2 peptide has an amino acid sequence that is at least 90% identical to the amino acid sequence of SEQ ID NO:1.

5. The method of any one of claims 1 to 3 claim 1, wherein the NEIL2 peptide is a functional fragment of SEQ ID NO:1.

6. The method of any one of claims 1 to 3 claim 1, wherein the NEIL2 peptide has an amino acid sequence of SEQ ID NO:1.

7. The method of claim 1, wherein the NEIL2 peptide is a fusion peptide or peptide complex comprising a targeting segment.

8. The method of claim 1, wherein the targeting segment is a lung cell targeting segment.

9. The method of claim 8, wherein the lung cell targeting segment has an amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

10. The method of claim 8, wherein the lung cell targeting segment has an amino acid sequence of SEQ ID NO:9.

11. The method of claim 1, further comprising a step of administering to the subject at least one additional antiviral therapeutic agent.

12. The method of claim 1, wherein the symptom is at least one of respiratory distress, organ dysfunction, systemic inflammatory response syndrome (SIRS), hypotension, tachycardia, dyspnea, ischemia, insufficient tissue perfusion, uncontrollable hemorrhage, multisystem organ failure or severe metabolism dysregulation.

13. The method of claim 1, wherein the virus is from Coronaviridae, Pneumoviridae, Orthomyxoviridae, Filoviridae, Flaviviridae, or Poxviridae families.

14. The method of claim 13, wherein the virus is a Coronavirus or Pneumovirus.

15. The method of claim 14, wherein the Coronavirus is a SARS-CoV-2 virus.

16. The method of claim 1, wherein the subject is a human subject.

17. The method of claim 1, wherein the NEIL2 peptide is administered to the subject at an amount from about 0.05 mg to about 0.5 mg peptide/kg body weight of said subject.

18. The method of claim 11, wherein the NEIL2 peptide and the additional antiviral therapeutic agent are administered to the subject simultaneously.

19. A method for treating a subject having a viral infection comprising the step of administering a therapeutically effective amount of a NEIL2 peptide or a composition comprising the NEIL2 peptide to the subject by inhalation or instillation.

20.-39. (canceled)

40. A method of treating a viral infection comprising:
identifying a subject at risk for a virus induced dysregulation of inflammatory response by measuring NEIL2 protein levels in a biological sample from a subject suspected of such a risk; and
administering a therapeutic composition comprising a NEIL2 peptide to the subject when the subject is identified as at risk for a virus induced dysregulation of inflammatory response when the NEIL2 protein levels are below a reference level for NEIL2 protein.

41.-57. (canceled)
